(19) United States
(12) Patent Application Publication
(10) Pub. No.: US 2024/0327886 A1
(43) Pub. Date: Oct. 3, 2024

(54) METHODS OF PRODUCING HMO BLEND PROFILES WITH LNFP-I AND LNT AS THE PREDOMINANT COMPOUNDS

(71) Applicant: DSM IP Assets B.V., Heerlen (DK)

(72) Inventors: Manos PAPADAKIS, Hørsholm (DK); Ted JOHANSON, Hørsholm (DK); Peter BECKER, Hørsholm (DK)

(21) Appl. No.: 18/561,233

(22) PCT Filed: May 17, 2022
(86) PCT No.: PCT/EP2022/063316

§ 371 Date: Nov. 15, 2023

(30) Foreign Application Priority Data
May 17, 2021 (DK) PA202170251

(51) Int. Cl. C12P 19/04; C07K 14/245; C12N 1/20; C12N 9/10; C12N 15/52; C12P 19/18; C12R 1/19

(52) U.S. Cl. CPC C12P 19/04; C07K 14/245; C12N 1/20; C12N 9/1051; C12N 15/52; C12P 19/18; C12R 2001/19; C12Y 204/01062; C12Y 204/01069; C12Y 204/01222

(57) ABSTRACT

This invention relates to a method of producing mixtures of various human milk oligosaccharides (HMOs) with unique HMO blend profiles, consisting predominantly of LNFP-I and LNT and of other HMOs in less significant amounts. The less abundant HMOs might be 2′-FL, LNT-II or DFL. The strategies for achieving specific HMO blends include strain engineering and fermentation methods.

### CROSS-REFERENCE TO RELATED APPLICATIONS

This application is a national stage entry pursuant to 35 U.S.C. § 371 of International Application No. PCT/EP2022/063316, filed on May 17, 2022, which claims priority to Denmark Application No. PA202170251, filed on May 17, 2021, the entire contents of all of which are hereby incorporated by reference in their entirety.

### SEQUENCE LISTING

This instant application contains a Sequence Listing which has been submitted in a ASCII text file via Patent Center and is hereby incorporated by reference in its entirety. Said text file, created on Nov. 14, 2023, is named 032991-8009 Sequence Listing.txt, and is 87,635 bytes in size.

### FIELD

This invention relates to a method of producing mixtures of various human milk oligosaccharides (HMOs) with unique HMO blend profiles, consisting predominantly of LNFP-I and LNT and of other HMOs in less significant amounts. The less abundant HMOs might be 2′-FL, LNT-II, or DFL. Several genetic engineering approaches have been applied to change the abundance of the different HMOs being produced by cells that over-express the colanic acid gene cluster and express a heterologous β-1,3-N-acetyl-glucosaminyltransferase, a β-1,3-galactosyltransferase and an α-1,2-fucosylfucosyltransferase. The strain engineering strategies to achieve this goal depend on the α-1,2-fucosylfucosyltransferase introduced in the host and comprise the overexpression of the native gene, lacY, coding the lactose permease LacY and the deletion of the glpR regulator, coding the DNA-binding transcriptional repressor GlpR, as well as the modulation of the lactose levels in the fermentation broth.

### BACKGROUND

Human milk represents a complex mixture of carbohydrates, fats, proteins, vitamins, minerals and trace elements. The by far most predominant fraction is represented by carbohydrates, which can be further divided into lactose and more complex oligosaccharides (Human Milk Oligosaccharides, HMO). Whereas lactose is used as an energy source, the complex oligosaccharides are not metabolized by the infant. The fraction of complex oligosaccharides accounts for up to 1/10 of the total carbohydrate fraction and consists of probably more than 150 different oligosaccharides. The occurrence and concentration of these complex oligosaccharides are specific to humans and thus cannot be found in large quantities in the milk of other mammals, like for example domesticated dairy animals.

To date, the structures of at least 115 HMOs have been determined, and considerably more are probably present in human milk. HMOs have become of great interest in the last decade, due to the discovery of their important functionality in human development. Besides their prebiotic properties, HMOs have been linked to additional positive effects, which expands their field of application. The health benefits of HMOs have enabled their approval for use in foods, such as infant formulas and foods, and for consumer health products.

To bypass the drawbacks associated with the chemical synthesis of HMOs, several enzymatic methods and fermentative approaches have been developed. Fermentation based processes have traditionally been developed for individual HMOs such as 2′-fucosyllactose (2′-FL), 3-fucosyllactose (3-FL), lacto-N-tetraose (LNT), lacto-N-neotetraose (LNnT), 3′-sialyllactose (3′-SL) and 6′-sialyllactose (6′-SL). Fermentation based processes typically utilize genetically engineered bacterial strains, such as recombinant Escherichia coli (E. coli) or yeast, such as Saccharomyces cerevisiae (S. cerevisiae) (see for example Bych et al Current Opinion in Biotechnology 56:130-137 and Lu et al 2021 ACS Synth. Biol. 10:923-938).

Biotechnological production, such as a fermentation process, of HMOs is a valuable, cost-efficient and large-scale approach to HMO manufacturing. It relies on genetically engineered bacteria constructed so as to express the glycosyltransferases needed for synthesis of the desired oligosaccharides and takes advantage of the bacteria's innate pool of nucleotide sugars as HMO precursors. At present, knowledge as to how to make composition of Lacto-N-fucopentaose I (LNFP-I)-containing blends and how to fine tune the levels of the different HMOs of the acquired blends either by genetic engineering or adjustment of fermentation parameters is nonexisting, because commercial fermentation process parameters for HMO manufacturing are normally kept secret and therefore the effects of e.g., fermentation parameters on LNFP-I blend compositions, or any other HMO blends, has not been described.

WO 2019/0011133 describes the identification of fucosyltransferases that can fucosylate LNT or LNnT to produce LNFP-I, LNFP-II, LNFP-III and LNFP-VI. Specifically, the use of FucT fucosyltransferases to produce LNFP-I are described.

WO 2019/123324 describes the formation of LNFP-I, there is however no indication of the molar % of LNFP-I or 2′FL constituted in the total amount of HMO formed.

### SUMMARY

The present disclosure targets biosynthetic production of HMO blends with specific ratios of the HMO(s) of interest in the blend, while the industrial focus normally is on producing pure HMOs, i.e., typically is to minimize HMO by-product levels and the consequential need to purify the HMO of interest in downstream processes.

Several genetic engineering approaches have been applied to change the abundance of the different HMOs being produced by cells that over-express the colanic acid gene cluster and express a heterologous β-1,3-N-acetyl-glucosaminyltransferase, a β-1,3-galactosyltransferase and an α-1,2-fucosylfucosyltransferase.

However, we observed that most genetic manipulations affected the ratio between LNFP-I and 2′-FL, and to a lesser extend the relative abundance of the precursor oligosaccharides, such as LNT and LNT-II, in the final HMO blend. This is particularly true for cells expressing the α-1,2-fucosyltransferase FutC from Helicobacter pylori (GenBank ID: WP_080473865.1, but with two additional amino acids (LG) at the C-terminus), where no genetic change could result in a final blend with LNFP-I being the most abundant and LNT the second most abundant HMO. Instead, any genetic modification in these cells resulted solely in minor or pronounced changes in the LNFP-I to 2′-FL ratio.

This invention highlights two principal ways of achieving unique and diverse blends of HMOs with LNFP-I and LNT as the predominant HMOs, namely strain engineering strategies and fermentation process strategies. The strain engineering strategies to achieve this goal comprise the manipulation of the following genetic traits of the HMO producer cell:
1. Over-expression the native gene lacY encoding the lactose permease LacY in cells expressing the α-1,2-fucosylfucosyltransferase Smob from Sulfuriflexus mobilis
2. Change in the regulatory landscape of the cell (deletion of the gene coding the GlpR regulator) in strains expressing the α-1,2-fucosylfucosyltransferase Smob from Sulfuriflexus mobilis

This disclosure enables the skilled person to produce specific HMO blends, with LNFP-I and LNT as the predominant HMOs, out of a broad diversity of possible blend compositions and tailor them to specific markets, customers and to achieve specific biological effects, while knowledge of the biological activity and function of specific HMOs and HMO mixtures is rapidly emerging.

The advantage is that the blends are manufactured by one producer strain and purified as a mixture of HMOs, hence, not mixed from individually purified HMOs produced by several producer strains. This gives a more sustainable manufacturing process; valuable HMOs are not discarded during the purification process and the conversion from carbon source to HMO product in fermentation is thus done at a much higher overall yield.

The fermentation process strategy in this disclosure includes modulation of the lactose levels in the fermentation broth to achieve a specific HMO blend profile with a given strain derived from strain engineering, in a highly predictable manner.

In its broadest aspect, the present disclosure relates to a method for the production of a human milk oligosaccharide (HMO) blend with LNFP-I and/or LNT as the predominant HMOs, the method comprising the steps of
(a) providing a genetically engineered cell capable of producing an HMO, wherein said cell expresses
   i) a heterologous β-1,3-N-acetyl-glucosaminyl-transferase protein as shown in SEQ ID NO: 1 [LgtA] or a functional homologue thereof having an amino acid sequence which is at least 80% identical to SEQ ID NO: 1 [LgtA]; and
   ii) a heterologous β-1,3-galactosyltransferase protein as shown in SEQ ID NO: 2 [GalTK] or a functional homologue thereof having an amino acid sequence which is at least 80% identical to SEQ ID NO: 2 [GalTK]; and
   iii) a heterologous α-1,2-fucosyltransferase protein as shown in SEQ ID NO: 3 [Smob] or 8 [FutC] or a functional homologue thereof having an amino acid sequence which is at least 80% identical to any one of SEQ ID NO: 3 [Smob] or 8 [FutC], and
   iv) a lactose permease protein as shown in SEQ ID NO: 4, or a functional homologue thereof having an amino acid sequence which is at least 80% identical to SEQ ID NO: 4, and
and further comprising
   v) a functional colanic acid gene cluster, and
   vi) a native or heterologous regulatory element, optionally in the form of an episomal element, for controlling the expression of i) ii) iii) and v), and
   vii) a native or heterologous regulatory element, optionally in the form of an episomal element, for increasing the expression of iv), and/or
   viii) a non-functional (or absent) gene product that normally binds to and represses the expression driven by vi)-vii)
(b) culturing the cell according to (a) in a suitable cell culture medium to express said nucleic acid sequences; and
(c) harvesting the HMO blend produced in step (b).

The term a non-functional (or absent) gene product that normally binds to and represses the expression driven by vi)-vii) in the present context relates to DNA binding sites upstream of the coding sequence of a gene of interest and specifically at the promoter region of this gene.

The regulatory element in vi-vii), such as a promoter, controls the expression of the mentioned glycosyltransferases (i-iii) and/or lactose permease (iv) and the colanic acid gene cluster (v), and this regulatory element should independently precede the coding sequence of i-v of the construct (promoter/regulatory element+coding sequence). The construct may be integrated into the genome, or it can be introduced into the cell in the form of a plasmid or another episomal element.

In another aspect, the present disclosure relates to a genetically engineered cell comprising:
a) one or more nucleic acid sequence(s) encoding a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
b) one or more nucleic acid sequence(s) encoding a heterologous β-1,3-galactosyltransferase protein as shown in SEQ ID NO: 2 or a functional homologue thereof having an amino acid sequence which is at least 80% identical to SEQ ID NO: 2; and
c) one or more nucleic acid sequence(s) encoding a heterologous α-1,2-fucosyltransferase protein as shown in SEQ ID NO: 3 or a functional homologue thereof having an amino acid sequence which is at least 80% identical to any one of SEQ ID NO: 3, and
d) one or more nucleic acid sequence(s) encoding one or more nucleic acid sequence(s) encoding a lactose permease protein as shown in SEQ ID NO: 4, or a functional homologue thereof having an amino acid sequence which is at least 80% identical to SEQ ID NO: 4, and
e) more than one nucleic acid sequence(s) encoding the proteins of the colanic acid gene cluster.

In an additional aspect, the disclosure relates to the use of a genetically engineered cell, or a nucleic acid construct according to the present disclosure, for the biosynthetic production of one or more Human Milk Oligosaccharides (HMOs), in particular a human milk oligosaccharide (HMO) blend with LNFP-I and LNT as the predominant HMOs.

### DETAILED DESCRIPTION

Various exemplary embodiments and details are described hereinafter, with reference to the figures and sequences when relevant. It should also be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

This invention enables the skilled person to produce specific HMO blends, with LNFP-I and/or LNT as the predominant HMOs. Markets are requesting a larger broad diversity of possible blend compositions, so they can tailor them to specific biological effects, because the knowledge of the biological activity and function of both specific HMOs and HMO mixtures is rapidly emerging.

The advantage here is that the blends are manufactured by one single strain and purified as a mixture of HMOs and hence, not mixed from individually purified HMOs. This gives a more sustainable manufacturing process; valuable HMOs are not discarded during the purification process and the conversion from carbon source to HMO product in fermentation is thus done at a higher overall yield.

In one or more exemplary embodiments, the methods described herein relates to the production of a human milk oligosaccharide (HMO) blend with LNFP-I and LNT as the predominant HMOs.

The LNFP-I and/or LNT Enzymes

For the production of human milk oligosaccharide (HMO) blend with LNFP-I and/or LNT as the predominant HMOs the genetically engineered cells comprise all the required enzymes to facilitate the production of a human milk oligosaccharide (HMO) blend with LNFP-I and/or LNT as the predominant HMOs. These enzymes may for example be
i) a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
ii) a heterologous β-1,3-galactosyltransferase protein as shown in SEQ ID NO: 2 or a functional homologue thereof having an amino acid sequence which is at least 80% identical to SEQ ID NO: 2; and
iii) a heterologous α-1,2-fucosyltransferase protein as shown in SEQ ID NO: 3 or a functional homologue thereof having an amino acid sequence which is at least 80% identical to any one of SEQ ID NO: 3, and
iv) a lactose permease protein as shown in SEQ ID NO: 4, or a functional homologue thereof having an amino acid sequence which is at least 80% identical to SEQ ID NO: 4.

The above enzymes can be exchanged by others with similar functionality. Especially, SEQ ID NO: 3, which can be exchanged with SEQ ID NO: 8. When using SEQ ID NO: 8, the level of lactose during culturing becomes an important fermentation parameter, as shown in Example 3.

In a preferred embodiment the invention, the level of lactose in the fermentation medium during the culturing of the genetically engineered cell in step (b) is below 15 g/L, when the genetically engineered cell comprises a heterologous α-1,2-fucosyltransferase protein as shown in SEQ ID NO: 8 or a functional homologue thereof having an amino acid sequence which is at least 80% identical to any one of SEQ ID NO: 8,

In one or more exemplary embodiments an over-expression of any of the protein(s) in i)-iv) is provided by increasing the copy number of the genes coding said protein(s).

### Heterologous β-1,3-N-Acetyl-Glucosaminyl-Transferase

A heterologous β-1,3-N-acetyl-glucosaminyl-transferase is any protein which comprises the ability of transferring the N-acetyl-glucosamine of UDP-N-acetyl-glucosamine to lactose. The β-1,3-N-acetyl-glucosaminyl-transferase used herein does not originate in the species of the genetically engineered cell i.e., the gene encoding the β-1,3-galactosyltransferase is of heterologous origin. The examples below use the heterologous β-1,3-N-acetyl-glucosaminyl-transferase named LgtA.

### IgtA Genes

The IgtA gene is a gene encoding a β-1,3-N-acetyl-glucosaminyl-transferase, and homologues of the gene are found in several bacterial species, wherein the gene is involved in the synthesis of the lacto-N-neo-tetraose structural element of the bacterial lipooligosaccharides.

In one or more exemplary embodiments, the IgtA gene is a nucleic acid sequence as shown in SEQ ID NO: 5 or is a functional homologue thereof having a nucleic acid sequence which is at least 70% identical, such as is at least 80%, such as at least 85%, such as at least 90%, such as at least 95% or such as at least 99% identical to SEQ ID NO: 5.

In one or more exemplary embodiments, LgtA a protein with an amino acid sequence of SEQ ID NO: 1, or a functional homologue thereof having an amino acid sequence which is at least 80%, such as at least 85%, such as at least 90%, such as at least 95% or such as at least 99% identical to SEQ ID NO: 1.

The expression of the β-1,3-N-acetyl-glucosaminyl-transferase, LgtA, can be modulated e.g., by varying the copy number of the gene encoding the β-1,3-N-acetyl-glucosaminyl-transferase, LgtA, which in turn may result in a shifted LNT/LNFP-1 ratio in the produced HMO blend. This is exemplified in example 4, where an additional copy of the LgtA gene (3 copies in total, under strong promoters) resulted in an enhanced LNT level and a reduced LNFP-I level (higher LNT/LNFP-I ratio in the produced HMO blend.

In one or more exemplary embodiments, at least two copies, such as at least 3 copies of the nucleic acid encoding the LgtA protein with an amino acid sequence of SEQ ID NO: 1, or a functional homologue thereof having an amino acid sequence which is at least 80%, such as at least 85%, such as at least 90%, such as at least 95% or such as at least 99% identical to SEQ ID NO: 1, is present in the genetically engineered cell.

Heterologous β-1,3-galactosyltransferase

A heterologous β-1,3-Galactosyltransferase is any protein that comprises the ability of transferring the Gal of UDP-Gal to a N-acetyl-glucosaminyl moiety. The β-1,3-galactosyltransferase used herein does not originate in the species of the genetically engineered cell i.e., the gene encoding the β-1,3-galactosyltransferase is of heterologous origin. The examples below use the heterologous β-1,3-galactosyltransferase named GalTK.

In one or more exemplary embodiments, GalTK is a protein with an amino acid sequence of SEQ ID NO: 2, or a functional homologue thereof having an amino acid sequence which is at least 80%, such as at least 85%, such as at least 90%, such as at least 95% or such as at least 99% identical to SEQ ID NO: 2.

galTK Genes

In one or more exemplary embodiments, the galTK gene is a nucleic acid sequence as shown in SEQ ID NO: 6 or is a functional homologue thereof having a nucleic acid sequence which is at least 70% identical, such as is at least 80%, such as at least 85%, such as at least 90%, such as at least 95% or such as at least 99% identical to SEQ ID NO: 6.

Heterologous α-1,2-fucosyltransferase

A α-1,2-fucosyltransferase is responsible for adding a fucose onto the galactose residue of the O-antigen repeating unit via an α-1,2 linkage.

In one or more exemplary embodiments, the heterologous α-1,2-fucosyltransferase is the Smob α-1,2-fucosyltransferase from Sulfuriflexus mobilis, (GenBank ID: WP_126455392.1) or a functional homologue thereof having an amino acid sequence which is at least 80% identical.

In one or more exemplary embodiments, the heterologous α-1,2-fucosyltransferase is the Smob α-1,2-fucosyltransferase from Sulfuriflexus mobilis with an amino acid sequence of SEQ ID NO: 3, or a functional homologue thereof having an amino acid sequence which is at least 80%, such as at least 85%, such as at least 90%, such as at least 95% or such as at least 99% identical to SEQ ID NO: 3.

In another exemplary embodiment, the heterologous α-1,2-fucosyltransferase is the FutC α-1,2-fucosyltransferase from Helicobacter pylori having the amino acid sequence according to SEQ ID NO: 8 (GenBank ID: WP_080473865.1) or a functional homologue thereof having an amino acid sequence which is at least 80% identical to SEQ ID NO: 8.

In one or more exemplary embodiments, the heterologous α-1,2-fucosyltransferase is the FutC α-1,2-fucosyltransferase from Helicobacter pylori, with an amino acid sequence of SEQ ID NO: 3, or a functional homologue thereof having an amino acid sequence which is at least 80%, such as at least 85%, such as at least 90%, such as at least 95% or such as at least 99% identical to SEQ ID NO: 3.

### α-1,2-Fucosyltransferase Smob Gene

The genetically engineered cells disclosed herein expressing the α-1,2-fucosyltransferase Smob from Sulfuriflexus mobilis (GenBank ID: WP_126455392.1) resulted in blends, where LNFP-I was the predominant HMO and LNT the second most abundant HMO. Specifically, as shown in the examples, through this disclosure, the person skilled in the art is able to pinpoint two specific genetic manipulations that lead to a simultaneous increase in LNT and a decrease in 2′-FL concentration in the final HMO mixture, while LNFP-I concentration was affected either positively or negatively, but only to a limited extend.

In one or more exemplary embodiments, the smob gene is a nucleic acid sequence as shown in SEQ ID NO: 7 or is a functional homologue thereof having a nucleic acid sequence which is at least 70% identical, such as is at least 80%, such as at least 85%, such as at least 90%, such as at least 95% or such as at least 99% identical to SEQ ID NO: 7.

α-1,2-fucosyltransferase futC Gene

In one or more exemplary embodiments, the futC gene is a nucleic acid sequence as shown in SEQ ID NO: 9 or is a functional homologue thereof having a nucleic acid sequence which is at least 70% identical, such as is at least 80%, such as at least 85%, such as at least 90%, such as at least 95% or such as at least 99% identical to SEQ ID NO: 9.

### Lactose Permease

Lactose permease is a membrane protein which is a member of the major facilitator superfamily and can be classified as a symporter, which uses the proton gradient towards the cell to transport β-galactosides such as lactose in the same direction into the cell. In HMO production lactose is the molecule being decorated to produce any HMO of interest and bioconversions happens in the cell interior. Thus, there is a desire to be able to import lactose into the cell, which can mainly be done by a certain activity of lactose permease, e.g., the native lacY copy under the control of a promoter.

In one or more exemplary embodiments, the lactose permease protein is as shown in SEQ ID NO: 4, or a functional homologue thereof having an amino acid sequence which is at least 80% identical, such as at least 85%, such as at least 90%, such as at least 95% or such as at least 99% identical to SEQ ID NO: 4.

In one or more exemplary embodiments, the lactose permease gene is a nucleic acid sequence as shown in SEQ ID NO: 10 or is a functional homologue thereof having a nucleic acid sequence which is at least 70% identical, such as is at least 80%, such as at least 85%, such as at least 90%, such as at least 95% or such as at least 99% identical to SEQ ID NO: 10.

### Lactose Permease Over-Expression

As shown in Example 1, the over-expression of the lacY gene coding lactose permease is used as a genetic tool to obtain a specific target composition of a HMO mixture comprising up to four HMOs, including LNFP-I, LNT, 2′-FL and LNT-II (in order of abundance).

The genetically engineered cells disclosed herein may comprise a regulatory element for increasing the expression of the native lactose permease protein, such as but not limited to Ribosome Binding Sites (RBSs). The RBSs may for example be the Shine-Dalgarno (SD) sequence. Mutations in the Shine-Dalgarno sequence can reduce or increase translation in prokaryotes. This change is due to a reduced or increased mRNA-ribosome pairing efficiency, as evidenced by the fact that compensatory mutations in the 3′-terminal 16S rRNA sequence can restore translation.

The regulatory element for increasing the expression of the native lactose permease protein could also be a promoter.

The genetically engineered cells disclosed herein may also comprise a heterologous episomal element for increasing the expression of the native lactose permease protein. This could for example be a plasmid-borne lacY gene.

The increased expression of the lactose permease may be achieved by direct integration of a copy of the lacY gene in the genome.

The increased expression of the lactose permease may be achieved by deleting a repressor of the lactose operon. An example of such being the lacl gene—UniProtKB-P03023 (LACI_ECOLI).

The genetically engineered cells disclosed herein may comprise a non-functional or absent gene product that normally binds to and represses the expression of the required enzymes to facilitate the production of a human milk oligosaccharide (HMO) blend with LNFP-I and LNT as the predominant HMOs.

As shown in the examples, the introduction of an additional copy of the lacY gene, which encodes the lactose permease, into smob-expressing cells resulted in a simultaneous marked increase in LNT concentration and a reduction in 2′-FL concentration. In this manner, the over-expression of the lacY gene in smob-expressing cells changed drastically the profile of the final HMO blend, with LNT—instead of 2′-FL—becoming the second most abundant HMO in the mixture. Regardless of lacY expression levels, LNFP-I remains the predominant HMO in blends generated by smob-expressing cells.

### Colanic Acid Gene Cluster

The colanic acid gene cluster of Escherichia coli K-12 is responsible for production of the extracellular polysaccharide colanic acid, a major oligosaccharide of the bacterial cell wall.

In one or more exemplary embodiments, the genetically modified cell in the methods disclosed herein may express the colanic acid gene cluster from its native genomic locus. The expression may be actively modulated. The expression can be modulated by swapping the native promoter with a promoter of interest, and/or increasing the copy number of the colanic acid genes coding said protein(s) by expressing the gene cluster (e.g. SEQ ID NO: 45 or a homologous sequence thereof) from another genomic locus, or episomally expressing the colanic acid gene cluster.

The colanic acid gene cluster can thus in one embodiment be a native genetic element of the HMO producing genetically modified cells used herein.

The colanic acid gene cluster as well as the heterologous genes that code a β-1,3-N-acetyl-glucosaminyl-transferase, a β-1,3-galactosyltransferase and an α-1,2-fucosyltransferase can further be introduced in the genetically modified cells used herein, in the form of PglpF-driven expression cassettes.

The deletion of the glpR gene (which codes the DNA-binding transcriptional repressor GlpR) can eliminate the GlpR-imposed repression of transcription from all PglpF promoters in the cell and in this manner enhance gene expression from all PglpF-based cassettes. Thus, the HMO content of the final blend can be affected in multiple ways. In the framework of the present disclosure, it was observed that deleting the glpR gene from the genetic background of futC-expressing cells (SEQ ID NO: 8) can increase the LNFP-I to 2′-FL ratio and, more importantly for this disclosure, also the LNT to 2′-FL ratio in the final HMO blend.

In one or more exemplary embodiments, the colanic acid gene cluster in the genetically modified cell of the invention (v)) may be expressed functionally.

### Expresses Functionally

In the present context, the term “expresses functionally” in relation to the colanic acid gene cluster should be understood as follows: the expression of the colanic acid (CA) gene cluster should provide the enzymes required for a functional GDP-fucose biosynthetic pathway.

The expression can be modulated by swapping the native promoter with a promoter of interest. The expression can also be modulated by increasing the copy number of the colanic acid genes encoding said protein(s). Episomally expressing the colanic acid gene cluster also affects the expression.

Thus, in one or more exemplary embodiments, the expression of the colanic acid gene cluster in v) is modulated by swapping the native promoter with a promoter of interest, and/or increasing the copy number of the colanic acid genes encoding said protein(s), or episomally expressing the colanic acid gene cluster.

In one or more exemplary embodiments, controlling the expression of the colanic acid gene cluster is modulated by swapping the native promoter with a promoter of interest, and/or increasing the copy number of the colanic acid genes coding said protein(s) by expressing the cluster from a different locus on the chromosome, or episomally expressing the colanic acid gene cluster. The individual genes in the CA gene cluster are described below.

### Gmd

The gmd gene encodes the protein GDP-mannose-4,6-dehydratase (UniProt accession nr P0AC88), which catalyzes the conversion of GDP-D-mannose to GDP-4-dehydro-6-deoxy-D-mannose. The protein is involved in the reaction that synthesizes GDP-L-fucose from GDP-alpha-D-mannose.

In one or more exemplary embodiments, the gmd gene is over-expressed.

wcaG

The wcaG gene, also known as fcl, encodes the protein GDP-L-fucose synthase (EC 1.1.1.271, UniProt accession nr P32055), which catalyses the two-step NADP-dependent conversion of GDP-4-dehydro-6-deoxy-D-mannose to GDP-fucose, involving an epimerase and a reductase reaction.

In one or more exemplary embodiments, the wcaG gene is over-expressed.

wcaH

The wcaH gene encodes the protein GDP-mannose mannosyl hydrolase (EC 3.6.1.-, UniProt accession nr P32056), that hydrolyzes both GDP-mannose and GDP-glucose.

In one or more exemplary embodiments, the wcaH gene is over-expressed.

wcaI

The wcaI gene encodes the colanic acid biosynthesis glycosyltransferase Wcal (UniProt accession nr P32057), and it catalyses the transfer of unmodified fucose to UPP-Glc (α-D-glucopyranosyl-diphosphoundecaprenol-glucose).

In one or more exemplary embodiments, the wcaI gene is over-expressed.

manB

The manB gene encodes the protein phosphomannomutase (EC 5.4.2.8, UniProt accession nr P24175), which is involved in the biosynthesis of GDP-mannose by catalysing conversion α-D-mannose-1-phosphate into D-mannose-6-phosphate. Thus, the expression level of manB regulates the formation of GDP-mannose.

In one or more exemplary embodiments, the manB gene is over-expressed.

manC

The manC gene encodes the protein mannose-1-phosphate guanylyltransferase (EC: 2.7.7.13, UniProt accession nr P24174), that is involved in the biosynthesis of GDP-mannose through synthesis of GDP-mannose from GTP and α-D-mannose-1-phosphate.

In one or more exemplary embodiments, the manC gene is over-expressed.

### Native Genomic Locus

In relation to the present disclosure, the term “native genomic locus”, in relation to the colanic acid gene cluster, relates to the original and natural position of the gene cluster in the native genome of the genetically engineered cell.

### Controlling the Expression

In the present context the term “controlling the expression” relates to gene expression where the transcription of a gene into mRNA and its subsequent translation into protein is controlled. Gene expression is primarily controlled at the level of transcription, largely as a result of binding of proteins to specific sites on DNA, such as but not limited to regulatory elements.

As described above, engineering strategy can be applied in multiple ways:
1) the copy number
2) the controlling the expression of any copy of these genes at the transcriptional or the translational level
3) the deletion of regulators that repress the expression of key genes in the HMO production process
4) the over-expression of regulators that activate and/or enhance the expression of key genes in the HMO production process

Increasing the gene copy number and/or the expression of genes coding the enzymes that are directly involved in the LNFP-I and 2′-FL biosynthetic pathways, including the synthesis of the activated sugars GDP-fucose, UDP-N-acetyl-glucosaminyl and UDP-Gal (donor sugars) and the decoration of lactose, LNT-II and LNT (acceptor sugars) to form, respectively, LNT-II or 2′-FL, LNT, and LNFP-I is desired.

### Over-Expression

A variety of molecular mechanisms ensures that genes are expressed at the appropriate level and under conditions of relevance to the applied production process. For instance, the regulation of transcription can be summarized into the following routes of influence; genetic (direct interaction of a control factor with the gene of interest), modulation and/or interaction of a control factor within the transcriptional machinery and epigenetic (non-sequence changes in DNA structure that influence transcription).

It is known that a reduction in gene expression below a critical threshold for any gene will result in a mutant phenotype, since such a defect essentially mimics either a partial or complete loss of function of the target gene, whereas increased expression of a native gene can be both beneficial or disruptive to a cell or organism.

Over-expression of a gene may be achieved directly by transcriptional activators that bind to key gene regulatory sequences to promote transcription or enhancers that constitute sequence elements positively affecting transcription, also termed regulatory elements as described below. Similarly, direct over-expression of a gene can be achieved by simply increasing its copy number in the genome, or replacing its native promoter with a promoter of higher strength or even modifying the sequence controlling the binding of the corresponding mRNA to the ribosomes, i.e., the Shine-Dalgarno sequence being present upstream of the gene's coding sequence.

Moreover, over-expression of a gene may also be achieved indirectly through the partial or full inactivation of transcriptional repressors that normally bind key regulatory sequences around the coding sequence of the gene of interest and thereby inhibit its transcription.

In embodiments, the endogenous or heterologous proteins, and/or genes are overexpressed and/or have an increased expression, which is obtained by increasing the copy number of the genes coding said protein(s).

Accordingly in embodiments, the expression of the colanic acid gene cluster is modulated by swapping the native promoter with a promoter of interest, and/or by increasing the copy number of the colanic acid genes coding said protein(s), or episomally expressing the colanic acid gene cluster or expressing it from a different locus on the chromosome.

Thus, in one or more exemplary embodiments, the over-expression of the protein(s) in i), ii) and iii) is provided by increasing the copy number of the genes coding said protein(s), and/or by choosing an appropriate element for or adding an extra genomic copy for iv) and/or v), and/or conferring a non-functional (or absent) gene product that normally binds to and repress the expression of any of i)-v).

### Increasing the Copy Number

Copy number variation is a type of structural variation: specifically, it is a type of duplication or multiplication of a considerable number of base pairs, which if representing a protein encoding gene will result in an increase of the number of genes encoding the same protein. Such variation can occur naturally in many species but can also be introduced by genetically modifying a host cell.

In one or more exemplary embodiments, expression is controlled by increasing the copy number of the desired gene(s). Copy numbers can be increased either by introducing a plasmid which has a high copy number in the cell or by introducing an additional copy of the gene into the genome of the host cell. In example, Example 4 shows that increasing the copy number of the β-1,3-N-acetyl-glucosaminyl-transferase, LgtA from two to three copies in a HMO blend producing strain, increases the amount of LNT in the produced HMO blend, compared to a strain comprising only two genetic copies of LgtA.

Thus, in one or more exemplary embodiments, the present disclosure relates to a method, wherein the overexpression of the β-1,3-N-acetyl-glucosaminyltransferase (i)) and the β-1,3-galactosyltransferase (ii)), exemplified e.g. by IgtA and galTK, in combination with the α-1,2-fucosylosyl-transferase (iii)) of SEQ ID NO: 3 [smob] or SEQ ID NO: [FutC] or a homologue thereof, is provided by increasing the copy number of the genes coding for said protein(s) and/or by choosing an appropriate regulatory element for the lactose permease (iv)).

### Regulatory Element

The genetically engineered cell according to the methods described herein may comprise regulatory elements enabling the controlled overexpression of endogenous or heterologous, and/or synthetic (recombinant) nucleic acid sequences.

In one or more exemplary embodiments, the heterologous regulatory element for controlling and increasing the expression of i)-v) in the method(s) described above is a promoter.

The term “regulatory element”, comprises promoter sequences, signal sequence, and/or arrays of transcription factor binding sites, that affect transcription and/or translation of a nucleic acid sequence operably linked to the regulatory element.

Regulatory elements are found at transcriptional and post-transcriptional levels and further enable molecular networks at those levels. For example, at the post-transcriptional level, the biochemical signals controlling mRNA stability, translation and subcellular localization are processed by regulatory elements. RNA binding proteins are another class of post-transcriptional regulatory elements and are further classified as sequence elements or structural elements. Specific sequence motifs that may serve as regulatory elements are also associated with mRNA modifications. A variety of DNA regulatory elements are involved in the regulation of gene expression and rely on the biochemical interactions involving DNA, the cellular proteins that make up chromatin, gene activators and repressors, and transcription factors.

In general, the transcriptional and translational regulatory sequences include, but are not limited to, promoter sequences, ribosomal binding sites, transcriptional start and stop sequences, translational start and stop sequences, binding sites for gene regulators and enhancer sequences.

Promoters and enhancers are the primary genomic regulatory components of gene expression. Promoters are DNA regions within 1-2 kilobases (kb) of a gene's transcription start site (TSS); they contain short regulatory elements (DNA motifs) necessary to assemble RNA polymerase transcriptional machinery. In bacterial and archaeal species is common to have a Shine-Dalgarno sequence downstream of the promoter, typically around 8 bases from the start codon. In addition, In addition, DNA regulatory elements located more distal to the TSS can contribute significantly to transcription. Such regions, often termed enhancers, are position-independent DNA regulatory elements that interact with site-specific transcription factors to establish cell type identity and regulate gene expression. Enhancers may act independently of their sequence context and at distances of several to many hundreds of kb from their target genes through a process known as looping. Because of these features, it is difficult to identify suitable enhancers and link them to their target genes based on DNA sequence alone.

The promoter, together with other transcriptional and translational regulatory nucleic acid sequences (also termed “control sequences”) is necessary to express a given gene or group of genes (an operon).

Identification of suitable promoter sequences that promotes expression of the specific gene of interest is a tedious task, which in many cases require laborious efforts. In relation to the present disclosure regulatory elements may or may not be post-translational regulators or it may or may not be translational regulators.

By choosing an appropriate regulatory element (e.g., promoter, enhancers and/or Shine-Dalgarno sequence) it is possible to affect the expression of a heterologous gene. The strength of a regulatory element, such as a promoter or Shine-Dalgarno sequence can be assed using a lacZ enzyme assay where β-galactosidase activity is assayed as described previously (see e.g., Miller J. H. Experiments in molecular genetics, Cold spring Harbor Laboratory Press, N Y, 1972). Briefly the cells are diluted in Z-buffer and permeabilized with sodium dodecyl sulfate (0.1%) and chloroform. The assays is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU.

Thus, in one embodiment of the invention the regulatory element comprises one or more elements capable of enhancing the expression, i.e., over-expression of the one or more heterologous nucleic acid sequence(s) according to the invention. The genetically engineered cells disclosed herein may comprise a heterologous regulatory element for controlling the expression of the heterologous β-1,3-N-acetyl-glucosaminyl-transferase, β-1,3-galactosyltransferase and/or the α-1,2-fucosyltransferase proteins.

In particular, the regulation of the expression levels of the encoded proteins can affect the formation of the different HMOs of the blend. In one embodiment regulatory elements of more than 10,000 MU, such as more than 12,000 MU, such as more than 15,000 MU is controlling the expression of 1,3-N-acetyl-glucosaminyltransferase and/or β-1,3-galactosyltransferase and/or α-1,2-fucosyltransferase and/or lactose permease and/or colanic acid gene cluster. Using strong promoters to control the expression of LacY resulted in an increase in the molar % of LNT in the blend, while deletion of the GlpR gene, which represses the PglpF promoters also enhanced the molar % of LNT in the blend (See FIGS. 1 and 2).

In another embodiment of the invention the regulatory element comprises one or more elements allowing appropriate control of the expression of the one or more heterologous nucleic acid sequence(s) according to the invention. In one embodiment regulatory elements of less than 10,000 MU, such as less than 8,000 MU, such as less than 6,000 MU such as less than 4,000 MU such as less than 2,000 MU is controlling the expression of 1,3-N-acetyl-glucosaminyltransferase and/or β-1,3-galactosyltransferase and/or α-1,2-fucosyltransferase and/or lactose permease and/or colanic acid gene cluster.

In that regard the regulatory element, regulating the expression of nucleic acid sequences and/or genes encoding one or more glycosyltransferases and/or sucrose hydrolyzing enzymes, and/or a PTS dependent sucrose utilization system and/or one or more native or heterologous MFS transporter proteins according to the invention, may be a promoter sequence.

In carrying out the methods as disclosed herein, different or identical promoter sequences may be used to drive transcription of different genes of interest integrated in the genome of the host cell or on episomal DNA.

### Native

In relation to the present disclosure, the term “native” refers to nucleic acid sequences originating from the genome of the genetically engineered cell according to the method of the invention. In that regard a nucleic acid sequence may be considered native if it originates from the E. coli K-12 strain, is not of heterologous origin and not a recombined nucleic acid sequence, with respect to the genetically engineered cell.

### Heterologous Expression

In the present disclosure, the term “heterologous” means that a nucleic acid encoding a protein has been introduced into a cell that does not normally make (i.e., express) that protein, such that the cell is capable of expressing the protein and is termed a genetically modified cell. Thus, heterologous refers to the fact that the expressed protein was initially cloned from or derived from a cell type or a species different from the recipient/host cell. The nucleic acid encoding the desired protein must be within a format that encourages the recipient cell to express the cDNA as a protein (i.e., it is put in an expression vector). Methods for transferring foreign genetic material into a recipient cell include transfection and transduction as well as crisper/cas. The choice of recipient cell type is often based on an experimental need to examine the protein's function in detail, and the most prevalent recipients, known as heterologous expression systems, are chosen usually because they are easy to transfer DNA into or because they allow for a simpler assessment of the protein's function.

### Heterologous Regulatory Element

A regulatory element may be endogenous or heterologous, and/or recombinant and/or synthetic nucleic acid sequences. In the present context, the term “heterologous regulatory element” is to be understood as a regulatory element that is not endogenously found in that genomic locus or heterologous to the original genetically engineered cell described herein. Hence, an endogenous PglpF promoter controlling the expression of a heterologous gene or an endogenous gene, such as lacY, substituting the native Plac promoter is according to the present invention considered to be heterologous regulatory element/promoter. The heterologous regulatory element may also be a recombinant regulatory element, wherein two or more non-operably linked native regulatory element(s) are recombined into a heterologous and/or synthetic regulatory element, examples of recombinant regulatory elements are PmglB_70UTR which combines the E. coli PmgIB promoter with the 70_UTR sequence of the E. coli PglpF promoter. The heterologous regulatory element, may be introduced into the genetically engineered cell using methods known to the person skilled in the art.

### Promoter Sequences

The regulatory element or elements regulating the expression of the genes and/or nucleic acid sequence(s), may comprise one or more promoter sequence(s), wherein the promoter sequence, is operably linked to the nucleic acid sequence of the gene of interest in that sense regulating the expression of the nucleic acid sequence of the gene of interest.

In one or more exemplary embodiments, the regulatory element is a promoter sequence.

In embodiments, the regulatory element for controlling and increasing the expression of endogenous or heterologous proteins, and/or genes is a promoter sequence.

In general, a promoter may comprise native, heterologous and/or synthetic nucleic acid sequences, and may be a recombinant nucleic acid sequence, recombining two or more nucleic acid sequences or same or different origin as described above, thereby generating a homologous, heterologous or synthetic nucleic promoter sequence, and/or a homologous, heterologous or synthetic nucleic regulatory element.

In one or more exemplary embodiments, the regulatory element of the genes and/or heterologous nucleic acid sequences of the genetically engineered cell comprises more than one native or heterologous promoter sequence.

In one or more exemplary embodiments, the regulatory element of the genetically engineered cell comprises a single promoter sequence.

In one or more exemplary embodiments, the regulatory element of the genes and/or heterologous nucleic acid sequences of the genetically engineered cell comprises two or more regulatory elements with identical promoter sequences.

In one or more exemplary embodiments, regulatory element of the genes and/or heterologous nucleic acid sequences of the genetically engineered cell comprises two or more regulatory elements with non-identical promoter sequences.

The regulatory architectures i.e., gene-by-gene distributions of transcription-factor-binding sites and identities of the transcription factors that bind those sites can be used multiple different growth conditions and there are more than 100 genes from across the E. coli genome, which acts as regulatory elements. Thus, any promoter sequence enabling transcription and/or regulation of the level of transcription, of one or more heterologous or native nucleic acid sequences that encode one or more proteins as described herein may be suitable.

In one or more exemplary embodiments, the regulatory element is selected from the group consisting of PBAD, Pxyl, PsacB, PxyIA, PrpR, PnitA, PT7, Ptac, PL, PR, PnisA, Pb, Pscr, Pscr_SD1, Pscr_SD7, PgatY_70UTR, PglpF, PglpF_SD1, PglpF_SD10, PglpF_SD2, PglpF_SD3, PglpF_SD4, PglpF_SD5, PglpF_SD6, PglpF_SD7, PglpF_SD8, PglpF_SD9, PglpF_B28, PglpF_B29, Plac_16UTR, Plac, PmglB_70UTR and PmglB_70UTR_SD4. A number of these regulatory elements are described in WO2019/123324 (hereby incorporate by reference).

In one or more exemplary embodiments, the regulatory element is selected from the group consisting of Pscr, PgatY_70UTR, PglpF, PglpF_SD1, PglpF_SD2, PglpF_SD3, PglpF_SD4, PglpF_SD5, PglpF_SD6, PglpF_SD7, PglpF_SD8, PglpF_SD9, PglpF_SD10, PglpF_B28, PglpF_B29, Plac and Plac_16UTR.

A wide selection of promoter sequences derived from the PglpF, PlgpA, PlgpT, PgatY, PmgIB and Plac promoter systems are described in detail WO2019123324 and WO2020255054 (hereby incorporated by reference).

In one or more exemplary embodiments, the regulatory element is a promoter selected from the group consisting of SEQ ID NO: 13 (PglpF), SEQ ID NO: 12 (PgatY_70UTR), SEQ ID NO: 27 (Plac), SEQ ID NO: 28 (PmglB_70UTR), SEQ ID NO: 11 (Pscr), or a variant thereof. In particular, a variant of PglpF or Plac as described in WO2019123324 or a variant of PmglB_70UTR as described in WO2020255054 is desired.

In one or more exemplary embodiments, the expression of the heterologous β-1,3-N-acetyl-glucosaminyltransferase and/or heterologous β-1,3-galactosyltransferase is obtained from a single copy and/or the regulatory element for expression of the heterologous β-1,3-N-acetyl-glucosaminyltransferase and/or heterologous β-1,3-galactosyltransferase has low or intermediate strength. The regulatory element with medium or low strength can be selected from the group consisting of PglpF_SD9 (SEQ ID NO: 23), PglpF_SD7 (SEQ ID NO: 21), PglpF_SD6 (SEQ ID NO: 20), PglpF_B28 (SEQ ID NO: 24), PglpF_B29 (SEQ ID NO: 25), Pscr (SEQ ID NO: 11 and Plac (SEQ ID NO: 27).

In one or more exemplary embodiments, the expression of the heterologous β-1,3-N-acetyl-glucosaminyltransferase and/or heterologous β-1,3-galactosyltransferase is obtained from two or more copies and/or the regulatory element for expression of the heterologous β-1,3-N-acetyl-glucosaminyltransferase and/or heterologous β-1,3-galactosyltransferase has high strength. The regulatory element with high strength can be is selected from the group consisting of PglpF (SEQ ID NO: 13) PglpF_SD10 (SEQ ID NO: 15), PglpF_SD8 (SEQ ID NO: 22), PglpF_SD5 (SEQ ID NO: 19), PglpF_SD4 (SEQ ID NO: 18), PgatY_70UTR (SEQ ID NO: 12), PmglB_70UTR (SEQ ID NO: 9) and PmgIB_70UTR_SD4 (SEQ ID NO: 9).

In preferred embodiments, the promoter sequence is selected from PglpF, Plac, PglpF_B29, PglpF_B28 and PmglB_70UTR.

In one or more exemplary embodiments, the regulatory element is selected from the group consisting of PglpF, PglpF_B29, and PglpF_B28.

In one or more exemplary embodiments, the regulatory element is selected from the group consisting of PglpF, Pscr, Plac, and PglpF_B28.

In further preferred embodiments the promoter sequence is selected from the group consisting of PglpF and PglpF_B28. In an embodiment the promoter sequence is PglpF. In another embodiment, the promoter sequence is PglpF_B28.

In a preferred exemplary embodiment, the promoter sequence comprised in the regulatory element for the regulation of the expression of the genes and/or heterologous nucleic acid sequences of the genetically engineered cell, encompasses the glpFKX operon promoter sequence, PglpF . . .

In one or more exemplary embodiments, the regulatory element is selected from the group consisting PglpF (SEQ ID NO: 13) or a variant thereof selected from PglpF_SD10 (SEQ ID NO: 15), PglpF_SD9 (SEQ ID NO: 23), PglpF_SD8 (SEQ ID NO: 22), PglpF_SD7 (SEQ ID NO: 21), PglpF_SD6 (SEQ ID NO: 20), PglpF_SD5 (SEQ ID NO: 19), PglpF_SD4 (SEQ ID NO: 18), PglpF_B28 (SEQ ID NO: 24) and PglpF_B29 (SEQ ID NO: 25).

In one or more exemplary embodiments, the promoter sequence comprised in the regulatory element for the regulation of the expression of the genes and/or heterologous nucleic acid sequences of the genetically engineered cell, encompasses the lac operon promoter sequence, Plac.

In one or more exemplary embodiments, the genetically engineered cell originates from the MDO strain (see “materials and methods”) and over-expresses a nucleic acid encoding the colanic acid gene cluster by a simple promoter swapping in front of the native colanic acid locus and/or the introduction of a second copy of this gene cluster at a different genomic locus [Plac (CA): PglpF_B28 (CA)].

In one or more exemplary embodiments, the regulatory element for the regulation of the expression of a recombinant gene included in the construct of the disclosure is the mglBAC; galactose/methyl-galactoside ABC transporter periplasmic binding protein promoter PmgIB or variants thereof such as but not limited to PmglB_70UTR, or PmglB_70UTR_SD4. Further PmgIB variants are described in as described in WO2020255054.

In one or more exemplary embodiments, the regulatory element for the regulation of the expression of a recombinant gene included in the construct of the disclosure is the gatYZABCD; tagatose-1,6-bisP aldolase promoter PgatY or variants thereof.

### Pscr

In one or more exemplary embodiments, the heterologous regulatory element is Pscr or variants thereof such as but not limited to SEQ ID NO: 11.

### Pscr_SD1

In one or more exemplary embodiments, the heterologous regulatory element is Pscr_SD1 or variants thereof such as but not limited to SEQ ID NO: 36.

### Pscr_SD7

In one or more exemplary embodiments, the heterologous regulatory element is Pscr_SD7 or variants thereof such as but not limited to SEQ ID NO: 37.

### PgatY_70UTR

In one or more exemplary embodiments, the heterologous regulatory element is PgatY_70UTR or variants thereof such as but not limited to SEQ ID NO: 12.

### PglpF

In one or more exemplary embodiments, the heterologous regulatory element is PglpF or variants thereof such as but not limited to SEQ ID NO: 13.

### PglpF_SD1

In one or more exemplary embodiments, the heterologous regulatory element is PglpF_SD1 or variants thereof such as but not limited to SEQ ID NO: 14.

### PglpF_SD10

In one or more exemplary embodiments, the heterologous regulatory element is PglpF_SD10 or variants thereof such as but not limited to SEQ ID NO: 15.

### PglpF_SD2

In one or more exemplary embodiments, the heterologous regulatory element is PglpF_SD2 or variants thereof such as but not limited to SEQ ID NO: 16.

### PglpF_SD3

In one or more exemplary embodiments, the heterologous regulatory element is PglpF_SD3 or variants thereof such as but not limited to SEQ ID NO: 17.

### PglpF_SD4

In one or more exemplary embodiments, the heterologous regulatory element is PglpF_SD4 or variants thereof such as but not limited to SEQ ID NO: 18.

### PglpF_SD5

In one or more exemplary embodiments, the heterologous regulatory element is PglpF_SD5 or variants thereof such as but not limited to SEQ ID NO: 19.

### PglpF_SD6

In one or more exemplary embodiments, the heterologous regulatory element is PglpF_SD6 or variants thereof such as but not limited to SEQ ID NO: 20.

### PglpF_SD7

In one or more exemplary embodiments, the heterologous regulatory element is PglpF_SD7 or variants thereof such as but not limited to SEQ ID NO: 21.

### PglpF_SD8

In one or more exemplary embodiments, the heterologous regulatory element is PglpF_SD8 or variants thereof such as but not limited to SEQ ID NO: 22.

### PglpF_SD9

In one or more exemplary embodiments, the heterologous regulatory element is PglpF_SD9 or variants thereof such as but not limited to SEQ ID NO: 23.

### PglpF_B28

In one or more exemplary embodiments, the heterologous regulatory element is PglpF_B28 or variants thereof such as but not limited to SEQ ID NO: 24.

### PglpF_B29

In one or more exemplary embodiments, the heterologous regulatory element is PglpF_B29 or variants thereof such as but not limited to SEQ ID NO: 25.

### Plac_16UTR

In one or more exemplary embodiments, the heterologous regulatory element is Plac_16UTR or variants thereof such as but not limited to SEQ ID NO: 26.

### Plac

In one or more exemplary embodiments, the heterologous regulatory element is Plac or variants thereof such as but not limited to SEQ ID NO: 27.

### PmglB_70UTR

In one or more exemplary embodiments, the heterologous regulatory element is PmglB_70UTR or variants thereof such as but not limited to SEQ ID NO: 28.

### PmglB_70UTR_SD4

In one or more exemplary embodiments, the heterologous regulatory element is PmglB_70UTR_SD4 or variants thereof such as but not limited to SEQ ID NO: 29.

### Episomal Element

The term “episomal element” refers to an extrachromosomal nucleic acid sequence, that can replicate autonomously or integrate into the genome of the genetically engineered cell. Thus, an episomal nucleic acid sequences may be a plasmid that can integrate into the chromosome of the genetically engineered cell, i.e., not all plasmids are episomal elements.

In one or more exemplary embodiments, episomal nucleic acid sequences may be a plasmid that is not integrated into the chromosome. In the present context, the episomal element refers to plasmid DNA sequences that carry an expression cassette of interest, with the cassette consisting of a promoter sequence, the coding sequence of the gene of interest and a terminator sequence.

In one or more exemplary embodiments, episomal nucleic acid sequences may be a plasmid with only a part of it being integrated into the chromosome. In the present context, the expression cassette resembles the one described above but it further comprises two DNA segments that are homologous to the DNA regions up- and downstream of the locus that the gene of interest is to be integrated.

### Activators

In one or more exemplary embodiment(s), the genetically engineered cell disclosed herein comprises an over-expressed gene product that enhances the expression of the gene(s) encoding the enzyme(s) required to facilitate the production of a human milk oligosaccharide(s) (HMOs) such as but not limited to LNFP-I, LNT, LNT-II and 2′-FL.

In one or more exemplary embodiments, the cell of the present disclosure may comprise an over-expressed gene product that binds to the regulatory element controlling the expression of the β-1,3-N-acetyl-glucosaminyltransferase, the β-1,3-galactosyltransferase and/or the α-1,2-fucosylosyl-transferase or CA cluster (vi)) or lactose permease (vii)), or regions upstream of the regulatory element of vi) or vii) and enhances the expression of the proteins β-1,3-N-acetyl-glucosaminyltransferase, the β-1,3-galactosyltransferase and/or the α-1,2-fucosylosyl-transferase and lactose permease (i) to iv)) or the colanic acid gene cluster (v)).

In one or more exemplary embodiments, the cell of the present disclosure may comprise an over-expressed gene product that binds to the regulatory element of vi) or vii) or regions upstream of the regulatory element of vi) or vii) and enhances the expression of the proteins of i) to iv) or the colanic acid gene cluster of v), and wherein the heterologous α-1,2-fucosyltransferase protein is Smob of SEQ ID NO: 3.

Moreover, the over-expression of regulators that enhances the expression of key genes in the HMO biosynthetic process is another plausible route towards the improvement of HMO titers in the final production cell, or the modification of the relative concentrations of different HMOs of a given blend. Thus, over-expression of the CRP-imposed enhancement of PglpF-driven gene expression of genes encoding enzymes catalysing key steps in the biosynthesis of different HMOs can result not only in higher total HMO titers, but also in multiple changes in the relative concentrations of the different HMOs that are present in LNFPI-containing HMO blends.

In one or more exemplary embodiments, the cell of the present disclosure may comprise an over-expressed gene product that binds to and enhances the expression of any of the β-1,3-N-acetyl-glucosaminyltransferase, the β-1,3-galactosyltransferase and/or the α-1,2-fucosylosyl-transferase, lactose permease and/or CA cluster (i)-v)), and wherein the heterologous α-1,2-fucosyltransferase protein is SEQ ID NO: 3 [Smob] or a functional homologue thereof.

In one or more exemplary embodiments, said gene product is the CAMP DNA-binding transcriptional dual regulator CRP.

### CRP

CRP belongs to the CRP-FNR superfamily of transcription factors. CRP regulates the expression of several of the E. coli genes, many of which are involved in catabolism of secondary carbon sources. Upon activation by cyclic-AMP, (CAMP) CRP binds directly to specific promoter sequences, the binding recruits the RNA polymerase through direct interaction, which in turn activates the transcription of the nucleic acid sequence following the promoter sequence leading to expression of the gene of interest.

Thus, over-expression of CRP may lead to an enhanced expression of a gene/nucleic acid sequence of interest. Amongst other functions, CRP exerts its function on the PglpF promoters, where it contrary to the repressor GlpR, activates promoter sequences of the PglpF family. In this way, over-expression of CRP in the genetically engineered cell of the present disclosure, promotes expression of genes that are regulated by promoters of the PglpF family.

As described above, the method according to the present disclosure relates addition of the nucleic acids encoding i) a heterologous β-1,3-N-acetyl-glucosaminyl-transferase protein and ii) a heterologous β-1,3-galactosyltransferase protein; and iii) a heterologous α-1,2-fucosyltransferase protein, and iv) a lactose permease protein. These proteins can be regulated by gene product that binds to and enhances the expression of i)-iv).

Thus, in one or more exemplary embodiments, the crp gene is over-expressed. The crp gene may encode a protein which is 100% identical to the amino acid sequence having the GenBank accession ID NP_417816 or a functional homologue thereof with is at least 70% identical, such as 80%, such as 90% such as 95% such as 98% identical to GenBank accession ID NP_417816.

Genetic engineering of GlpR and/or CRP as suggested in the present disclosure in HMO blend producing strains is beneficial for the overall production of LNT by these strains.

### Repressors

As shown in Example 2, the deletion of the glpR gene coding the DNA-binding transcriptional repressor GlpR is used as a genetic tool to obtain a specific target composition of a HMO mixture comprising up to four HMOs, including LNFP-I, LNT, LNT-II and 2′-FL (in order of abundance).

In one or more exemplary embodiments, the cell may have a non-functional (or absent) gene product(s) that would normally bind to and repress the expression of any of i)-v) or regions upstream of vi-viii) and represses the expression of i) to v).

In one or more exemplary embodiments, the colanic acid gene cluster may be expressed from an extra copy that is integrated at a non-native locus and under the control of the PglpF promoter or a variant thereof.

Moreover, the deletion of regulators that repress the expression of key genes in the HMO biosynthetic process is another plausible route towards the improvement of HMO titers in the final production cell, or the modification of the relative concentrations of different HMOs of a given blend. Thus, elimination of the GlpR-imposed repression of PglpF-driven gene expression of genes encoding enzymes catalysing key steps in the biosynthesis of different HMOs can result not only in higher total HMO titers, but also in multiple changes in the relative concentrations of the different HMOs that are present in LNFPI-containing HMO blends.

In one or more exemplary preferred embodiments, the method according to the present disclosure comprise a cell further comprising non-functional (or absent) gene product that binds to and represses the expression of any of i)-v), and wherein the heterologous α-1,2-fucosyltransferase protein is SEQ ID NO: 3 [Smob].

In one or more exemplary preferred embodiments, the method according to the present disclosure comprise a cell further comprising non-functional (or absent) gene product that binds to and represses the expression of any of i)-v), and wherein the heterologous α-1,2-fucosyltransferase protein is SEQ ID NO: 8 [FutC].

In one or more exemplary embodiments, said gene product is the DNA-binding transcriptional repressor GlpR.

### GlpR

GlpR belongs to the DeoR family of transcriptional regulators and acts as the repressor of the glycerol-3-phosphate regulon, which is organized in different operons. This regulator is part of the glpEGR operon, yet it can also be constitutively expressed as an independent (glpR) transcription unit. In addition, the operons regulated are induced when Escherichia coli is grown in the presence of inductor, glycerol, or glycerol-3-phosphate (G3P), and the absence of glucose. In the absence of inductor, this repressor binds in tandem to inverted repeat sequences that consist of 20-nucleic acid-long DNA target sites.

The term “non-functional or absent” in relation to the glpR gene refers to the inactivation of the glpR gene by complete or partial deletion of the corresponding nucleic acid sequence from the bacterial genome (e.g., SEQ ID NO: 44 or variants thereof encoding glpR capable of downregulating glpF derived promoters). The glpR gene can also be rendered non-functional by introducing mutations into the coding sequence which introduces stop codons, frameshifts or amino acid mutations that affects the DNA binding to the regulatory element. The glpR gene encodes the DNA-binding transcriptional repressor GlpR. In this way promoter sequences of the PglpF family are upregulated in the genetically engineered cell, due to deletion of the repressor gene that would otherwise downregulate the PglpF promoters.

As described above, the method according to the present disclosure relates addition of the nucleic acids encoding i) a heterologous β-1,3-N-acetyl-glucosaminyl-transferase protein and ii) a heterologous β-1,3-galactosyltransferase protein; and iii) a heterologous-1,2-fucosyltransferase protein, and iv) a lactose permease protein. These proteins can be regulated by gene product that normally binds to and represses the expression of i)-iv).

In one or more exemplary embodiments, the gene product is the transcriptional repressor GlpR.

In embodiments of the present invention, the genetically modified cell of the present invention comprises a non-functional (or absent) gene encoding the transcriptional repressor GlpR, which once functional binds to and represses the expression driven by the beforementioned regulatory elements, such as but not limited to PglpF promoter sequences.

In one or more exemplary embodiments, the glpR gene is deleted.

The deletion of the glpR gene from the genetic background of smob-expressing cells had a similar effect as the over-expression of the lacY gene, with LNT becoming the second most abundant HMO after LNFP-I. The colanic acid gene cluster as well as the heterologous genes that encode a β-1,3-N-acetyl-glucosaminyl-transferase, a β-1,3-galactosyltransferase and an α-1,2-fucosyltransferase were introduced in the E. coli host in the form of expression cassettes that enable gene expression under the control of the PglpF promoter, which is known to be repressed by the GlpR regulator. Thus, the deletion of the glpR gene could eliminate the GlpR-imposed repression of transcription from all PglpF promoters in the cell and in this manner enhance gene expression from all PglpF-based cassettes.

The increase in LNT concentration and decrease in 2′-FL titer in the final blend that is observed with glpR deletion, is surprising, since the expression of the colanic acid gene cluster and all 3 heterologous genes is PglpF-driven and it is thus expected to be affected similarly by the absence of the GlpR regulator. However, given the higher formation of LNT and the lower observed 2′-FL and LNFP-I titers, the glpR deletion is seemingly having a more positive impact on the expression of the β-1,3-N-acetyl-glucosaminyl-transferase and the β-1,3-galactosyltransferase rather than the expression of the α-1,2-fucosyltransferase or the colanic acid gene cluster. Alternatively, a plausible explanation for the observed increase in LNT, but not 2′-FL and LNFP-I concentration, in the final blend, which is generated by the cell that lacks the glpR gene, lies in the fact that the expression levels of LgtA (β-1,3-N-acetyl-glucosaminyl-transferase) and GalTK (β-1,3-galactosyltransferase)—but not Smob (α-1,2-fucosyltransferase) or the colanic acid gene cluster (responsible for GDP-fucose synthesis)—were limiting for the achievement of higher HMO titers. This limitation can be though overcome by the deletion of the glpR gene.

### Sugar Efflux Transporter

Over the past decade several new and efficient sugar efflux transporter proteins have been identified, each having specificity for different recombinantly produced HMOs and development of recombinant cells expressing said proteins are advantageous for high scale industrial HMO manufacturing. Sugar transport relates to the transport of a sugar, such as, but not limited to, an oligosaccharide.

In example 4, it is shown how engineering a Smob-expressing cells by introducing a few selected sugar efflux transporter proteins as well as increasing the expression levels of the fucosyltransferase or β-1,3-N-acetyl-glucosaminyl-transferase proved to be two efficient genetic modifications that can markedly modulate the order of abundance of the first and second most abundant HMO of the final HMO blend from LNFP-I>LNT to LNT>LNFP-1.

Thus, the genetically engineered cell(s) described herein, may also comprise a recombinant nucleic acid encoding a sugar efflux transporter. A sugar efflux transporter may for example enhance the level of an HMO in a method as described herein.

Influx and/or efflux transport of one/or more HMOs, from the cytoplasm or periplasm of a genetically engineered cell as described herein to the production medium and/or from the production medium to the cytoplasm or periplasm is disclosed. In one or more exemplary embodiments, the genetically engineered cell further comprises a gene product that acts as a sugar efflux transporter.

A polypeptide, expressed in the genetically engineered cell as disclosed herein, capable of transporting one or more HMOs from the cytoplasm or periplasm to the production medium and/or from the production medium to the cytoplasm or periplasm of a genetically engineered cell, is a polypeptide capable of sugar transport.

Thus, in the present context, sugar transport can mean efflux and/or influx transport of sugar, such as, but not limited to, an HMO.

Thus, in one or more exemplary embodiments, the genetically engineered cell according to the method described herein further comprises a gene product that acts as a sugar efflux transporter. The gene product that acts as a sugar efflux transporter may be encoded by a recombinant nucleic acid sequence that is expressed in the genetically engineered cell. The recombinant nucleic acid sequence encoding a sugar efflux transporter, may be integrated into the genome of the genetically engineered cell.

### MFS Transporters

Exemplary sugar efflux transporters are a subspecies of the Major Facilitator Superfamily proteins. The MFS transporters facilitate the transport of molecules, such as but not limited to sugars like oligosaccharides, across the cellular membranes.

By the term “Major Facilitator Superfamily (MFS)” is meant a large and exceptionally diverse family of the secondary active transporter class, which is responsible for transporting a range of different substrates, including sugars, drugs, hydrophobic molecules, peptides, organic ions, etc.

The term “MFS transporter” in the present context means, a protein that facilitates transport of an oligosaccharide, preferably, an HMO, through the cell membrane, preferably transport of an HMO/oligosaccharide synthesized by the genetically engineered cell as described herein from the cell cytosol to the cell medium. Additionally, or alternatively, the MFS transporter may also facilitate efflux of molecules that are not considered HMO or oligosaccharides, such as lactose, glucose, cell metabolites and/or toxins.

As shown in Example 4, it was possible-strictly by incorporation of one or more MFS transporters—to re-design the abundance of the LNT and LNFP-I blend composition quite remarkably. Example 4 demonstrates how inclusion of the MFS transporter Nec alone or in combination with YberC enhances the level of LNT in the blend becoming the most predominant HMO in the blend. The inclusion of YberC increases the level of LNT in the blend slightly, while the LNFP-I level was maintained at the same level as the control strain. Additional genetic modifications can therefore be used to modulate the LNT: LNFP-I molar ratio between the two extremities.

In one or more exemplary embodiments, the genetically engineered cell further comprises a recombinant nucleic acid encoding one or more sugar transport protein(s) and/or MFS transport protein.

In one or more exemplary embodiments, the sugar efflux transporter and/or MFS transport protein is selected from the group consisting of Bad, Nec, YberC, Fred, Vag and Marc.

In one or more presently preferred exemplary embodiments, the genetically sugar efflux transporter is YberC and/or Nec.

### Bad

The MFS transporter protein identified herein as “Bad protein” or “Bad transporter” or “Bad”, interchangeably, has the amino acid sequence of SEQ ID NO: 38; The amino acid sequence identified herein as SEQ ID NO: 38 is an amino acid sequence which is 100% identical to the amino acid sequence having the GenBank accession ID WP_017489914.1.

In one or more exemplary embodiments, the sugar efflux transporter and/or MFS transport protein is Bad. In a further embodiment the sugar efflux transporter has the amino acid sequence of SEQ ID NO: 38 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 38.

### Nec

The MFS transporter protein having the amino acid sequence of SEQ ID NO: 39 is identified herein as “Nec protein” or “Nec transporter” or “Nec”, interchangeably; a nucleic acid sequence encoding Nec protein is identified herein as “Nec coding nucleic acid/DNA” or “nec gene” or “nec”; The amino acid sequence identified herein as SEQ ID NO: 39 is the amino acid sequence which is 100% identical to the amino acid sequence having the GenBank accession ID WP_092672081.1.

In one or more exemplary embodiments, the sugar efflux transporter and/or MFS transport protein is Nec. In a further embodiment the sugar efflux transporter has the amino acid sequence of SEQ ID NO: 39 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 39.

As shown in example 4, expression of Nec in combination with the alpha-1,2-fucosyltransferase, Smob, results in an enhanced LNT production, compared to a cell not expressing Nec. In addition, example 4 shows that the level of LNT may be further enhanced when combining expression of Nec and Smob, with an enhanced expression of the β-1,3-N-acetyl-glucosaminyl-transferase, LgtA.

In one or more presently preferred exemplary embodiments, the sugar efflux transporter is Nec.

### YberC

The MFS transporter protein having the amino acid sequence of SEQ ID NO: 40 is identified herein as “YberC protein” or “YberC transporter” or “YberC”, interchangeably; a nucleic acid sequence encoding YberC protein is identified herein as “YberC coding nucleic acid/DNA” or “yberC gene” or “yberC”; The amino acid sequence identified herein as SEQ ID NO: 40 is the amino acid sequence which is 100% identical to the amino acid sequence having the GenBank accession ID EEQ08298.1.

In one or more exemplary embodiments, the sugar efflux transporter and/or MFS transport protein is YberC. In a further embodiment the sugar efflux transporter has the amino acid sequence of SEQ ID NO: 40 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 40.

As shown in example 4, expression of YberC in combination with the MFS transporter Nec and the alpha-1,2-fucosyltransferase, Smob, results in an HMO blend with both LNT and LNFP-1.

In one or more presently preferred exemplary embodiments, the cell comprises a recombinant nucleic acid sequence encoding the sugar transport protein(s) YberC and/or Nec.

### Fred

The MFS transporter protein having the amino acid sequence of SEQ ID NO: 41 is identified herein as “Fred protein” or “Fred transporter” or “Fred”, interchangeably; a nucleic acid sequence encoding freed protein is identified herein as “Fred coding nucleic acid/DNA” or “fred gene” or “fred”; The amino acid sequence identified herein as SEQ ID NO: 41 is the amino acid sequence which is 100% identical to the amino acid sequence having the GenBank accession ID WP_087817556.1.

In one or more exemplary embodiments, the sugar efflux transporter and/or MFS transport protein is Fred. In a further embodiment the sugar efflux transporter has the amino acid sequence of SEQ ID NO: 41 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 41.

### Vag

The MFS transporter protein having the amino acid sequence of SEQ ID NO: 42 is identified herein as “Vag protein” or “Vag transporter” or “Vag”, interchangeably; a nucleic acid sequence encoding Vag protein is identified herein as “Vag coding nucleic acid/DNA” or “vag gene” or “vag”; The amino acid sequence identified herein as SEQ ID NO: 42 is the amino acid sequence which is 100% identical to the amino acid sequence having the GenBank accession ID WP_048785139.1.

In one or more exemplary embodiments, the sugar efflux transporter and/or MFS transport protein is Vag. In a further embodiment the sugar efflux transporter has the amino acid sequence of SEQ ID NO: 42 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 42.

### Marc

The MFS transporter protein having the amino acid sequence of SEQ ID NO: 43 is identified herein as “Marc protein” or “Marc transporter” or “Marc”, interchangeably; a nucleic acid sequence encoding marc protein is identified herein as “Marc coding nucleic acid/DNA” or “marc gene” or “Marc”; The amino acid sequence identified herein as SEQ ID NO: 43 is the amino acid sequence which is 100% identical to the amino acid sequence having the GenBank accession ID WP_060448169.1.

In one or more exemplary embodiments, the sugar efflux transporter and/or MFS transport protein is Marc. In a further embodiment the sugar efflux transporter has the amino acid sequence of SEQ ID NO: 43 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 43.

In one or more exemplary embodiments, a sugar efflux transporter functional homologue having an amino acid sequence which is at least 70% identical, such as at least 80% identical, such as at least 85% identical, such as at least 90% identical, such as at least 95% identical or such as at least 99% identical to any one of SEQ ID NOs: 38 to 43.

One embodiment of the present disclosure relates to a genetically engineered cell comprising:
a) one or more nucleic acid sequence(s) encoding a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
b) one or more nucleic acid sequence(s) encoding a heterologous β-1,3-galactosyltransferase protein as shown in SEQ ID NO: 2 or a functional homologue thereof having an amino acid sequence which is at least 80% identical to SEQ ID NO: 2; and
c) one or more nucleic acid sequence(s) encoding a heterologous α-1,2-fucosyltransferase protein as shown in SEQ ID NO: 3 or a functional homologue thereof having an amino acid sequence which is at least 80% identical to any one of SEQ ID NO: 3, and
d) one or more nucleic acid sequence(s) encoding one or more nucleic acid sequence(s) encoding a lactose permease protein as shown in SEQ ID NO: 4, or a functional homologue thereof having an amino acid sequence which is at least 80% identical to SEQ ID NO: 4, and
e) more than one nucleic acid sequence(s) encoding the proteins of the colanic acid gene cluster; and
f) one or more nucleic acid sequence(s) encoding a MFS transporter selected from
   i) Nec with the amino acid sequence of SEQ ID NO: 39 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 39, and/or
   ii) YberC with the amino acid sequence of SEQ ID NO: 40 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 40.

One embodiment of the present disclosure relates to a genetically engineered cell comprising:
a) one or more nucleic acid sequence(s) encoding a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
b) one or more nucleic acid sequence(s) encoding a heterologous β-1,3-galactosyltransferase protein as shown in SEQ ID NO: 2 or a functional homologue thereof having an amino acid sequence which is at least 80% identical to SEQ ID NO: 2; and
c) one or more nucleic acid sequence(s) encoding a heterologous α-1,2-fucosyltransferase protein as shown in SEQ ID NO: 3 or a functional homologue thereof having an amino acid sequence which is at least 80% identical to any one of SEQ ID NO: 3, and
d) one or more nucleic acid sequence(s) encoding one or more nucleic acid sequence(s) encoding a lactose permease protein as shown in SEQ ID NO: 4, or a functional homologue thereof having an amino acid sequence which is at least 80% identical to SEQ ID NO: 4, and
e) more than one nucleic acid sequence(s) encoding the proteins of the colanic acid gene cluster; and
f) one or more nucleic acid sequence(s) encoding a MFS transporter selected from
   i) Nec with the amino acid sequence of SEQ ID NO: 39 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 39, and/or
   ii) YberC with the amino acid sequence of SEQ ID NO: 40 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 40; and
g) a non-functional (or absent) gene encoding the transcriptional repressor GlpR.

The genetically modified cell may further comprise:
i) a native or heterologous regulatory-optionally episomal-element for controlling the expression of a) b) c), e) and f), and
ii) a native or heterologous regulatory-optionally episomal-element for increasing the expression of d) where the regulatory element is the same or different than the element in i) and/or

Preferably, the genetically engineered cell comprises two or three copies of the β-1,3-N-acetyl-glucosaminyl-transferase gene in a).

### Nucleic Acid Constructs

An aspect of the present disclosure is the provision of a nucleic acid construct. The nucleic acid construct may comprise at least a nucleic acid sequence encoding
i) a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
ii) a heterologous β-1,3-galactosyltransferase protein as shown in SEQ ID NO: 2 or a functional homologue thereof having an amino acid sequence which is at least 80% identical to SEQ ID NO: 2; and
iii) a heterologous α-1,2-fucosyltransferase protein as shown in SEQ ID NO: 3 or 8 or a functional homologue thereof having an amino acid sequence which is at least 80% identical to any one of SEQ ID NO: 3 or 8, and

The nucleic acid construct also comprises at least a nucleic acid sequence encoding
i) a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
ii) a heterologous β-1,3-galactosyltransferase protein as shown in SEQ ID NO: 2 or a functional homologue thereof having an amino acid sequence which is at least 80% identical to SEQ ID NO: 2; and
iii) a heterologous α-1,2-fucosyltransferase protein as shown in SEQ ID NO: 3 or 8 or a functional homologue thereof having an amino acid sequence which is at least 80% identical to any one of SEQ ID NO: 3 or 8, and
iv) a lactose permease protein as shown in SEQ ID NO: 4, or a functional homologue thereof having an amino acid sequence which is at least 80% identical to SEQ ID NO: 4.

The nucleic acid construct may comprise at least one regulatory element that facilitates the functional expression of the colanic acid gene cluster from its native genomic locus.

The nucleic acid construct may further comprise one or more regulatory element for controlling the expression of i)-iii). The regulatory element(s) may also control the expression of the colanic acid gene cluster. The regulatory element(s) may be a native or heterologous or episomal.

The nucleic acid construct may further comprise a heterologous regulatory or episomal element for increasing the expression of iv) a lactose permease protein as shown in SEQ ID NO: 4, or a functional homologue thereof having an amino acid sequence which is at least 80% identical to SEQ ID NO: 4

The nucleic acid construct may further comprise a non-functional (or absent) gene product that normally binds to and represses the expression of the regulatory element(s).

The nucleic acid construct can be a recombinant nucleic acid sequence. By the term “recombinant nucleic acid sequence”, “recombinant gene/nucleic acid/DNA encoding” or “coding nucleic acid sequence” used interchangeably is meant an artificial nucleic acid sequence (i.e., produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

### A Recombinant Nucleic Acid Sequence

The recombinant nucleic sequence may be a coding DNA sequence e.g., a gene, or non-coding DNA sequence e.g., a regulatory DNA, such as a promoter sequence.

Accordingly, in one exemplified embodiment the invention relates to a nucleic acid construct comprising a coding nucleic sequence, i.e., recombinant DNA sequence of a gene of interest, e.g., a fucosyltransferase gene, and a non-coding regulatory DNA sequence, e.g., a promoter DNA sequence, e.g., a recombinant promoter sequence derived from the promoter sequence of lac operon or an glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. operably linked refers to the functional relationship of a transcriptional regulatory sequence to a transcribed sequence. For example, a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the invention may be a part of the vector DNA, in another embodiment the construct it is an expression cassette/cartridge that is integrated in the genome of a host cell.

Accordingly, the term “nucleic acid construct” means an artificially constructed segment of nucleic acid, in particular a DNA segment, which is intended to be ‘transplanted’ into a target cell, e.g., a bacterial cell, to modify expression of a gene of the genome or express a gene/coding DNA sequence which may be included in the construct.

Integration of the nucleic acid construct of interest comprised in the construct (expression cassette) into the bacterial genome can be achieved by conventional methods, e.g., by using linear cartridges that contain flanking sequences homologous to a specific site on the chromosome, as described for the attTn7-site (Waddell C. S. and Craig N. L., Genes Dev. (1988) February; 2 (2): 137-49.); methods for genomic integration of nucleic acid sequences in which recombination is mediated by the Red recombinase function of the phage λ or the RecE/RecT recombinase function of the Rac prophage (Murphy, J Bacteriol. (1998); 180 (8): 2063-7; Zhang et al., Nature Genetics (1998) 20:123-128 Muyrers et al., EMBO Rep. (2000) 1 (3): 239-243); methods based on Red/ET recombination (Wenzel et al., Chem Biol. (2005), 12 (3): 349-56.; Vetcher et al., Appl Environ Microbiol. (2005); 71 (4): 1829-35); or positive clones, i.e., clones that carry the expression cassette, can be selected e.g., by means of a marker gene, or loss or gain of gene function.

Integration can be at one or more sites in the genome of the host cell. If integrated at one genomic site in the host cell the recombinant nucleic acids can either be under the control of a single regulatory element forming an operon or under control of individual regulatory elements. Alternatively, the recombinant nucleic acids can be integrated in several places in the genome of the host cell under the control of individual regulatory elements.

In one or more exemplary embodiments, the present disclosure relates to a recombinant nucleic acid shown in SEQ ID NO: 5-7 or 9-10, or a functional homologue thereof having a sequence which is at least 70% identical to SEQ ID NO: 5-7 or 9-10, such as at least 71% identical, at least 72% identical, at least 73% identical, at least 74% identical, at least 75% identical, at least 76% identical, at least 77% identical, at least 78% identical, at least 79% identical, at least 80% identical, at least 75% identical, at least 75% identical, at least 75% identical, at least 75% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical at least 96% identical, at least 97% identical, at least 98% identical, or at least 99% identical.

### Sequence Identity

The term “sequence identity of [a certain] %” in the context of two or more nucleic acid or amino acid sequences means that the two or more sequences have nucleic acids or amino acid residues in common in the given percent, when compared and aligned for maximum correspondence over a comparison window or designated sequences of nucleic acids or amino acids (i.e., the sequences have at least 90 percent (%) identity). Percent identity of nucleic acid or amino acid sequences can be measured using a BLAST 2.0 sequence comparison algorithm with default parameters, or by manual alignment and visual inspection (see e.g., http://www.ncbi.nlm.nih.gov/BLAST/). This definition also applies to the complement of a test sequence and to sequences that have deletions and/or additions, as well as those that have substitutions. An example of an algorithm that is suitable for determining percent identity, sequence similarity and for alignment is the BLAST 2.2.20+algorithm, which is described in Altschul et al. Nucl. Acids Res. 25, 3389 (1997). BLAST 2.2.20+ is used to determine percent sequence identity for the nucleic acids and proteins of the disclosure. Software for performing BLAST analyses is publicly available through the National Center for Biotechnology Information (http://www.ncbi.nlm.nih.gov/). Examples of commonly used sequence alignment algorithms are
CLUSTAL Omega (http://www.ebi.ac.uk/Tools/msa/clustalo/),
EMBOSS Needle (http://www.ebi.ac.uk/Tools/psa/emboss_needle/),
MAFFT (http://mafft.cbrc.jp/alignment/server/), or
MUSCLE (http://www.ebi.ac.uk/Tools/msa/muscle/).

Preferably, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment).

Preferably, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16:276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides×100)/(Length of Alignment-Total Number of Gaps in Alignment).

### Functional Homologue

A functional homologue of a protein/nucleic acid sequence as described herein is a protein/nucleic acid sequence with alterations in the genetic code, which retain its original functionality. A functional homologue may be obtained by mutagenesis. The functional homologue should have a remaining functionality of at least 50%, such as 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of SEQ ID NOs: 1-10 or 30-45, should ideally be able to participate in the HMO production, in terms of HMO yield, purity, reduction in biomass formation, viability of the genetically engineered cell, robustness of the genetically engineered cell according to the disclosure, or reduction in consumables.

### Genetically Engineered Cell

The present disclosure also relates to a genetically engineered cell comprising
a) one or more nucleic acid sequence(s) encoding a heterologous β-1,3-N-acetyl-glucosaminyl-transferase, a β-1,3-galactosyltransferase and an α-1,2-fucosyltransferase protein according to SEQ ID NO: 1-3, or 8 or a functional homologue thereof having an amino acid sequence which is at least 70% identical to SEQ ID NO: 1-3, or 8
b) one or more nucleic acid sequence(s) encoding a lactose permease protein as shown in SEQ ID NO: 4, or a functional homologue thereof having an amino acid sequence which is at least 80% identical to SEQ ID NO: 4.
c) more than one nucleic acid sequence(s) encoding the proteins of the colanic acid gene cluster.

In an aspect of the invention the genetically engineered cell comprises:
a) one or more nucleic acid sequence(s) encoding a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
b) one or more nucleic acid sequence(s) encoding a heterologous β-1,3-galactosyltransferase protein as shown in SEQ ID NO: 2 or a functional homologue thereof having an amino acid sequence which is at least 80% identical to SEQ ID NO: 2; and
c) one or more nucleic acid sequence(s) encoding a heterologous α-1,2-fucosyltransferase protein as shown in SEQ ID NO: 3 or a functional homologue thereof having an amino acid sequence which is at least 80% identical to any one of SEQ ID NO: 3, and
d) one or more nucleic acid sequence(s) encoding one or more nucleic acid sequence(s) encoding a lactose permease protein as shown in SEQ ID NO: 4, or a functional homologue thereof having an amino acid sequence which is at least 80% identical to SEQ ID NO: 4, and
e) more than one nucleic acid sequence(s) encoding the proteins of the colanic acid gene cluster.

The genetically engineered cell may also comprise
i) a native or heterologous regulatory element—for controlling the expression of a), b) and/or c), and
ii) a native or heterologous regulatory element for increasing the expression of d), and/or
iii) a non-functional (or absent) gene product that normally binds to and represses the expression driven i) and/or ii).
iv)

In one embodiment the regulatory element of ii) is different than the regulatory element of i). This can be useful to facilitate a balance between the substrate, lactose, and the expression level of the glycosyltransferases.

Preferably, the genetically engineered cell further comprises a recombinant nucleic acid sequence encoding a sugar efflux transporter capable of changing the ratio of LNFP-I and LNT.

A “genetically engineered cell” as used herein is understood as a cell which has been transformed or transfected, by a recombinant nucleic acid sequence. Accordingly, a “genetically engineered cell” is in the present context understood as a host cell which has been transformed or transfected by a recombinant nucleic acid sequence.

The genetically engineered cell according to the present invention may apply modifications to enzymes, transporters, regulatory elements, activator and repressors as described in the sections above. In one or more exemplary embodiments, the cell is capable of producing one or more HMO(s) selected from the group consisting of 2′-FL, LNT-II, LNT, LNFP-I and DFL.

In one or more exemplary embodiments, the genetically engineered cell is capable of producing one or more HMO(s) selected from the group consisting of 2′-FL, LNT-II, LNT and LNFP-I.

In one or more exemplary embodiments, the predominant HMO produced by the genetically engineered cell is LNFP-I and/or LNT. Preferably, the molar % of LNT and LNFP-I combined is above 75%, such as above 80% of the total HMO, wherein the HMO blend has molar % of LNT between 10% to 70% and LNFP-I between 30% to 95% of the total HMO.

The genetically engineered cell may be any cell useful for HMO production including mammalian cell lines. Preferably, the host cell is a unicellular microorganism of eucaryotic or prokaryotic origin.

Appropriate microbial cells that may function as a host cell include yeast cells, bacterial cells, archaebacterial cells, algae cells, and fungal cells.

The genetically engineered cell (host cell) may be e.g., a bacterial or yeast cell. In one preferred embodiment, the genetically engineered cell is preferably a prokaryotic cell, such as a bacterial cell.

### Bacterial Host Cells

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities.

Non-limiting examples of bacterial host cells that are suitable for recombinant industrial production of an HMO(s) according to the invention could be Erwinia herbicola (Pantoea agglomerans), Citrobacter freundii, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of an HMO(s) according to the invention could be yeast cells, such as Komagataella phaffii, Kluyveromyces lactis, Yarrowia lipolytica, Pichia pastoris, and Saccaromyces cerevisiae or filamentous fungi such as Aspargillus sp, Fusarium sp or Thricoderma sp, exemplary species are A. niger, A. nidulans, A. oryzae, F. solani, F. graminearum and T. reesei.

In one or more exemplary embodiments, the genetically engineered cell is S. cerevisiae or P. pastoris.

In one or more exemplary embodiments, the genetically engineered cell is Pichia pastoris.

In one or more exemplary embodiments, the genetically engineered cell is S. cerevisiae

In one or more exemplary embodiments, the genetically engineered cell is selected from the group consisting of E. coli, C. glutamicum, L. lactis, B. subtilis, S. lividans, P. pastoris, and S. cerevisiae.

In one or more exemplary embodiments, the genetically engineered cell is selected from the group consisting of B. subtilis, S. cerevisiae and Escherichia coli.

In one or more exemplary embodiments, the genetically engineered cell is B. subtilis.

In one or more exemplary embodiments, the genetically engineered cell is Escherichia coli.

In one or more exemplary embodiments, the invention relates to a genetically engineered cell, wherein the cell is derived from the E. coli K-12 or DE3 strain.

### Culturing

In the present context, culturing refers to the process by which cells are grown under controlled conditions, generally outside their natural environment, thus a method used to cultivate, propagate and grow a large number of cells.

The terms culturing and fermentation are used interchangeably.

A further aspect of the present disclosure relates to a method for the production of a human milk oligosaccharide (HMO) blend with LNFP-I and LNT as the predominant HMOs, the method comprising the steps of
(a) providing a genetically engineered cell capable of producing an HMO, wherein said cell expresses
   i) a heterologous β-1,3-N-acetyl-glucosaminyl-transferase protein as shown in SEQ ID NO: 1 [LgtA] or a functional homologue thereof having an amino acid sequence which is at least 80% identical to SEQ ID NO: 1 [LgtA]; and
   ii) a heterologous β-1,3-galactosyltransferase protein as shown in SEQ ID NO: 2 [GalTK] or a functional homologue thereof having an amino acid sequence which is at least 80% identical to SEQ ID NO: 2 [GalTK]; and
   iii) a heterologous α-1,2-fucosyltransferase protein as shown in SEQ ID NO: 3 [Smob] or a functional homologue thereof having an amino acid sequence which is at least 80% identical to SEQ ID NO: 3 [Smob], and
   iv) a lactose permease protein as shown in SEQ ID NO: 4, or a functional homologue thereof having an amino acid sequence which is at least 80% identical to SEQ ID NO: 4, and further comprising,
   v) a functional colanic acid gene cluster, and
   vi) a native or heterologous regulatory element, optionally in the form of an episomal element, for controlling the expression of i) ii) iii) and v), and
   vii) a native or heterologous regulatory element, optionally in the form of an episomal element, for increasing the expression of iv), and/or
   viii) a non-functional (or absent) gene product that normally binds to and represses the expression driven by vi)-vii)
(b) culturing the cell according to (a) in a suitable cell culture medium to express said nucleic acid sequences; and
(c) harvesting the HMO blend produced in step (b).

In an alternative embodiment the heterologous α-1,2-fucosyltransferase protein in iii) is exchanged with the heterologous α-1,2-fucosyltransferase as shown in SEQ ID NO: 8 [FutC] or a functional homologue thereof having an amino acid sequence which is at least 80% identical to SEQ ID NO: 8 [FutC].

### Cell Culture Medium

In the present context, a growth medium or culture medium is a liquid or gel designed to support the growth of microorganisms, cells, or small plants. The medium comprises an appropriate source of energy and may comprise compounds which regulate the cell cycle. The culture medium may be semi-defined, i.e., containing complex media compounds (e.g., yeast extract, soy peptone, casamino acids, etc.), or it may be chemically defined, without any complex compounds. Exemplary suitable medias are provided in experimental examples.

In one or more exemplary embodiments, the culturing media is minimal media.

In one or more exemplary embodiments, the culturing media is supplemented with one or more energy and carbon sources selected form the group containing glycerol, sucrose, glucose and fructose.

In one or more exemplary embodiments, the culturing media is supplemented with one or more energy and carbon sources selected form the group containing glycerol, sucrose and glucose.

In one or more exemplary embodiments, the culturing media is supplemented with glycerol, sucrose and/or glucose.

In one or more exemplary embodiments, the culturing media is supplemented with glycerol and/or glucose.

In one or more exemplary embodiments, the culturing media is supplemented with sucrose and/or glucose.

In one or more exemplary embodiments, the culturing media is supplemented with glycerol and/or sucrose.

In one or more exemplary embodiments, the culturing media is supplemented only with sucrose.

In one or more exemplary embodiments, the culturing media contains sucrose as the sole carbon and energy source.

### Sucrose Fermentation

In one or more exemplary embodiments, the genetically engineered cell may comprise a PTS-dependent sucrose utilization transport system and/or a recombinant nucleic acid sequence encoding a heterologous polypeptide capable of hydrolysing sucrose into fructose and glucose.

Such cells are capable of utilizing sucrose as carbon and energy source. For example, the culturing step according to step b) of the method(s) disclosed herein comprises a two-step sucrose feeding, with a second feeding phase by continuously adding to the culture an amount of sucrose that is less than that added continuously in a first feeding phase so as to slow the cell growth and increase the content of product produced in the high cell density culture.

The feeding rate of sucrose added continuously to the cell culture during the second feeding phase may be around 30-40% less than that of sucrose added continuously during the first feeding phase.

During both feeding phases, lactose can be added continuously, preferably with sucrose in the same feeding solution, or sequentially.

Optionally, the culturing further comprises a third feeding phase when considerable amount of unused acceptor remained after the second phase in the extracellular fraction.

Then the addition is sucrose is continued without adding the acceptor, preferably with around the same feeding rate set for the second feeding phase until consumption of the acceptor.

In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons.

In one or more exemplary embodiments the polypeptide encoded by the scrYA operon are polypeptides with an amino acid sequence according to SEQ ID NOs: 30-31 [scrY and scrA] or a functional homologue of any one of SEQ ID NOs: 30-31 [scrY and scrA], which amino acid sequence is at least 80% identical to any one of SEQ ID NO: 30-31 [scrY and scrA].

In one or more exemplary embodiments the polypeptide encoded by the scrBR operon are polypeptides with an amino acid sequence according to SEQ ID NOs: 32-33 [scrB and scrR] or a functional homologue of any one of SEQ ID NOs: 32-33 [scrB and scrR], which amino acid sequence is at least 80% identical to any one of SEQ ID NOS: 32-33 [scrB and scrR].

In one or more exemplary embodiments, the polypeptide capable of hydrolyzing sucrose into fructose and glucose is selected from the group consisting of SEQ ID NOs: 34-35 [SacC_Agal and Bff], or a functional homologue of any one of SEQ ID NOs: 34-35 [SacC_Agal and Bff], which amino acid sequence is at least 80% identical to any one of SEQ ID NO: 34-35 [SacC_Agal and Bff].

### Level of Lactose During Culturing

For many LNFP-I producing strains, derived by the above strain engineering strategies, one single main fermentation parameter was found to significantly impact the composition of the resulting HMO blend profiles in a predictable and unique manner.

Modulation of fermentation parameter may enable profiles with a pre-determined composition within narrow ranges for each of the HMOs contained in the blends. Hence, the level of lactose during fermentation was found to have a very big impact on the composition of a 4-HMO blend for one family of LNFP-I producing strains (see Example 3).

As shown in Example 3, it was possible-only by variation of the lactose levels from low to high—to re-design the order of abundance of the second, third and fourth most abundant HMO in LNFP-I blends quite remarkably from 2′-FL>LNT>LNT-II to LNT>LNT-II>2′-FL. Example 3, demonstrates how modulation of the lactose level during fermentation can be used to obtain a specific target composition of a HMO mixture comprising up to four HMOs in significant quantities of LNFP-I, 2′-FL, LNT and LNT-II.

Therefore, this disclosure shows how lactose addition during fermentation can be advantageously used to modulate the composition of the HMO blend produced by strains MP5 and MP6.

In one or more exemplary embodiments, the level of lactose in the culturing media is modulated.

Thus, in one or more exemplary embodiments, the level of lactose during the culturing of the genetically engineered cell is modulated from low to high. In the present context, and as shown in FIG. 3, this resulted in the following lactose concentration ranges: high lactose process 30-80 g/L, low lactose process 0-15 g/L.

Thus, in one or more exemplary embodiments, a high level of lactose level relates to 30-80 g/L, such as but not limited to 30-40 g/L, 30-50 g/L, 30-60 g/L, 30-70 g/L, 40-50 g/L, 40-60 g/L, 40-70 g/L, 40-80 g/L, 50-60 g/L, 50-70 g/L, 50-80 g/L, 60-70 g/L, 60-80 g/L, 35-50 g/L, 35-60 g/L, 35-70 g/L, 35-75 g/L, 35-80 g/L, 45-55 g/L, 45-75 g/L, 55-65 g/L, 55-75 g/L, 55-80 g/L, 65-75 g/L, or 65-80 g/L.

In one embodiment of the invention the level of lactose in the fermentation medium during the culturing of the genetically engineered cell in step (b) is between 30-80 g/L. The advantage of this is a LNFP-I blend with more LNT compared to 2′-FL (see table 4).

Thus, in one or more exemplary embodiments, a low level of lactose level relates to 0-15 g/L, such as but not limited to 0-5 g/L, 0-7.5 g/L, 0-10 g/L, 0-12.5 g/L, 2.5-5 g/L, 2.5-7.5 g/L, 2.5-10 g/L, 2.5-12.5 g/L, 2.5-15 g/L, 5-7.5 g/L, 5-10 g/L, 5-12.5 g/L, 5-15 g/L, 7.5-10 g/L, 7.5-12.5 g/L, 7.5-15 g/L, 10-12.5 g/L, 10-15 g/L, or 12.5-15 g/L.

In one embodiment of the invention the level of lactose the fermentation medium during the culturing of the genetically engineered cell in step (b) is below 15 g/L. This will result in a LNFP-I blend with both 2′-FL and LNT, where 2′FL is more prevalent than LNT (see table 4).

No other fermentation parameters besides the lactose concentration was found to have such big impact on the HMO blend compositions for LNFP-I producing strains.

Since it is desired to convert the E. coli host to a cell factory for HMO production, two of our major focus areas in the applied rational genetic engineering program are to a) increase the lactose import into the cell and b) to increase the expression of the enzymes directly involved in the different HMO biosynthetic pathways. It is a rather plausible assumption that increasing the import of lactose (i.e., the primary substrate for HMO synthesis) into the cell can boost the biosynthesis of the LNT-II, which is a precursor sugar for LNT and LNFP-I synthesis, and/or the synthesis of the HMO 2′-FL. Therefore, increasing the mRNA levels and presumably the protein levels of the lactose permease can lead to more LacY molecules in the plasma membrane of the cell, which in turn can increase the intracellular concentration of lactose, which is the primary substrate for HMO synthesis.

More substrate in the cell interior can affect the relative abundance of different HMOs in multiple manners, mainly depending on the activities and the kinetics of the expressed glycosyltransferases. The over-expression of the lacY gene is thus a powerful genetic tool for widening the spectrum of HMO blends that can be generated.

### Harvesting

The term “harvesting” in the context relates to collecting the produced HMO(s) following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation) include extraction thereof from the biomass (i.e the production cells).

After recovery from fermentation, HMO(s) are available for further processing and purification.

### Human Milk Oligosaccharide (HMO)

In the context of the disclosure, the term “oligosaccharide” means a saccharide polymer containing a number of monosaccharide units. The number of monosaccharide units are in the range of 3 to 15, such as in the range of 3 to 10 such as in the range of 3 to 6. In some embodiments, preferred oligosaccharides are saccharide polymers consisting of three or four monosaccharide units, i.e., trisaccharides or tetrasaccharides or pentasaccharides or hexasaccharides. Preferable oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl units, and this core structure can be substituted by an alpha-L-fucopyranosyl and/or an alpha-N-acetyl-neuraminyl (sialyl) moiety.

In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-triose 2 (LNT-2) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3′-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In the context of the present disclosure lactose is not regarded as an HMO species.

### Specific HMO Blends

The term “blend” or “HMO blend” refers to a mixture of two or more HMOs and/or HMO precursors, such as but not limited to HMOs selected from LNT, LNnT, LNH, LNT-II, LNnH, para-LNH, para-LNnH, 2′-FL, 3FL, DFL, LNFP I, LNDFH-I, LNFP II, LNFP III, LNFP V, F-LNnH, DF-LNH I, DF-LNH II, DF-LNH I, DF-para-LNH, DF-para-LNnH, 3′-SL, 6′-SL, FSL, F-LST a, F-LST b, F-LST c, LST a, LST b, LST c and DS-LNT. The HMO blends as described herein are obtained at the end of fermentation and not by mixing purified HMOs or HMOs produced by different fermentation batches. An HMO blend is the composition of HMOs produced during or at the end of fermentation, the HMO blend at the end of fermentation may also be termed the final HMO blend. The blend of HMOs may be subjected to downstream purification, however with the purpose to maintain a blend of HMO's with similar ratios as in the blend of HMOs obtained after fermentation. It is envisioned that no additional HMOs are added to the blend following the purification.

In one or more exemplary embodiments, the “HMO blend” referred to herein relates to a mixture of two or more HMOs and/or HMO precursors selected from the group consisting of LNT, LNT-II, LNnH, para-LNH, 2′-FL, DFL, and LNFP I. Preferably, the HMO blend, or the major components of the HMO blend are produced from a single production strain.

This disclosure highlights two principal ways of achieving unique and diverse blends of HMOs with LNFP-I and LNT as the predominant HMOs, namely strain engineering strategies and fermentation process strategies. The strain engineering strategies to achieve this goal comprise the manipulation of the following genetic traits of the HMO producer cell
1. Choice of the introduced α-1,2-fucosyltransferase
2. Extent of the expression of the colanic acid gene cluster
3. Extent of the expression or copy number of the β-1,3-N-acetyl-glucosaminyltransferase
4. Introduction of one or more sugar transporter(s) of the Major Facilitator Superfamily (MFS)
5. Change in the regulatory landscape of the cell e.g., deletion of the glpR regulator

The fermentation process strategies in this disclosure include modulation of the lactose levels in the fermentation broth to achieve a specific HMO blend profile with a given strain derived from strain engineering, in a highly predictable manner.

The HMO products produced by the methods disclosed herein can also be given in ratios. The “ratio” as described herein is understood as the ratio between two amounts of HMOs, such as but not limited to the amount of one divided by the amount of the other. As shown in Table 4, the following ranges can be found for the high lactose process in molar %: LNFP-I/HMO [60-68], LNT/HMO [21-27], LNT-II/HMO [6-9], 2′-FLHMO [4-6], and the following ranges were found for the low lactose process in molar %: LNFP-I/HMO [66-70], 2′-FL/HMO [25-30], LNT/HMO [3-4], LNT-II/HMO [1-1.5].

Hence, lactose concentration can be powerful control tool to achieve a pre-determined, desired profile of 3-4 major HMOs during the production of HMO blends containing predominantly LNFP-I.

### Predominant HMO

The term “predominant” is used herein to define a single HMO species being more than 70 molar % of the total amount of harvested HMOs, such as but not limited to more than 71 molar %, 72 molar %, 73 molar %, 74 molar %, 75 molar %, 76 molar %, 77 molar %, 78 molar %, 79 molar %, 80 molar %, 81 molar %, 82 molar %, 83 molar %, 84 molar %, 85 molar %, 86 molar %, 87 molar %, 88 molar %, 89 molar %, 90 molar %, 91 molar %, 92 molar %, 93 molar %, 94 molar %, 95 molar %, 96 molar %, 97 molar %, 98 molar %, 99 molar %, or 99.5 molar % of the total HMO.

The same definition applies to a blend of HMOs, meaning that a blend of for example two HMOs are “predominant”, when the blend is more than 70 molar % of the total amount of harvested HMOs.

In one embodiment of the invention the molar % of LNT and LNFP-I combined is above 75%, such as above 80% of the total HMO.

In one embodiment of the invention, following fermentation with a strain described herein the HMO blend has molar % of LNFP-I between 65 molar % and 95 molar % of the total HMO and LNT between 2.4 molar % and 35 molar % of the total HMO. Preferably, the HMO blend of the invention has a molar % of LNT between 10% to 70% and LNFP-I between 30% to 95% of the total HMO.

### Ratio

Thus, in one or more exemplary embodiments, the ratio of LNFP-I: LNT harvested in step c) is in the range of 25:1-1:3, such as 25:1-1:1, such as but not limited to 22:1, 18:1, 15:1, 12:1, 10:1, 7:1, 5:1, 4:1, 3:1, 2:1 1.1, 2:3 or 1:3. In an alternative embodiment LNT is absent from said product.

In one or more exemplary embodiments, the ratio of LNFP-I: LNT harvested in step c) is in the range of 10:1 to 1:3, such as 10:1 to 1:1, such as but not limited to 9:1, 8:1, 7:1, 6:1, 5:1, 4:1, 3:1, 2.1, 1:1, 2:3 or 1:3.

In one or more presently preferred exemplary embodiments, the ratio of LNFP-I: LNT in the harvested HMOs is 10:1, 5:1, 3:1, 5:2, 2:3 or 1:3, 2:3 or 1:3

### Absent HMOs

In one or more exemplary embodiments, LNT-II is absent (not present) from the harvested HMOs.

In one or more exemplary embodiments, 2′-FL is absent (not present) from the harvested HMOs.

An HMO is considered absent from the harvested HMOs in step c) in the methods described herein, when the amount of said HMO constitute less than 1 molar % of the total amount of harvested HMOs, such as but not limited to less than 0.9 molar %, less than 0.1 molar %, less than 0.01 molar %, less than 0.001 molar % of the total amount of harvested HMOs.

### Use of a Genetically Engineered Cell

The disclosure also relates to any commercial use of the genetically engineered cell(s) or the nucleic acid construct(s) disclosed herein. The genetically engineered cell(s) or the nucleic acid construct(s) comprise nucleic acid sequences encoding at least 3 different heterologous proteins, such as but not limited to
i) a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
ii) a heterologous β-1,3-galactosyltransferase protein as shown in SEQ ID NO: 2 or a functional homologue thereof having an amino acid sequence which is at least 80% identical to SEQ ID NO: 2; and
iii) a heterologous α-1,2-fucosyltransferase protein as shown in SEQ ID NO: 3 or 8 or a functional homologue thereof having an amino acid sequence which is at least 80% identical to any one of SEQ ID NO: 3 or 8.

The genetically engineered cell(s) or the nucleic acid construct(s) may further comprise a nucleic acid encoding a lactose permease protein as shown in SEQ ID NO: 4, or a functional homologue thereof having an amino acid sequence which is at least 80% identical to SEQ ID NO: 4.

The genetically engineered cell(s) or the nucleic acid construct(s) may further express actively the colanic acid gene cluster from its native or any other genomic locus.

The genetically engineered cell(s) or the nucleic acid construct(s) may comprise a heterologous regulatory element for controlling the expression of i), ii) and iii).

The genetically engineered cell(s) or the nucleic acid construct(s) may also comprise a (heterologous) regulatory or episomal element for increasing the expression of the lactose permease.

The genetically engineered cell(s) or the nucleic acid construct(s) may comprise a non-functional (or absent) gene product that normally binds to and represses the expression of the β-1,3-N-acetyl-glucosaminyl-transferase, β-1,3-galactosyltransferase, α-1,2-fucosyltransferase and/or lactose permease.

In one or more exemplary embodiments, the genetically engineered cell or the nucleic acid construct is used in the manufacturing of one or more HMOs. The one or more HMOs can be selected from the group consisting of 2′-FL, LNT-II, LNT, LNnT, LNFP-I, and DFL. In a presently preferred embodiment, the one or more HMOs is/are selected from the group consisting of 2′-FL, LNT-II, LNT and LNFP-I.

In one or more exemplary embodiments, the genetically engineered cell and/or the nucleic acid construct is used in the manufacturing of more than one HMO(s), wherein the one or more HMOs is/are selected from the group consisting LNT-II, LNT and LNFP-I.

In another exemplified embodiment, the genetically engineered cell and/or the nucleic acid construct according to the invention, is used in the manufacturing of more than one HMO(s), wherein the HMOs predominantly are LNFP-I and LNT.

In one or more exemplary embodiments, the genetically engineered cell and/or the nucleic acid construct is used in the manufacturing of an HMO blend consisting of LNFP-I and LNT.

### Manufacturing of HMOs

To produce one or more HMOs, the genetically engineered cells as described herein are cultivated according to the procedures known in the art in the presence of a suitable carbon and energy source, e.g., glucose, glycerol or sucrose, and a suitable acceptor, e.g., lactose or any HMO, and the produced HMO blend is harvested from the cultivation media and the microbial biomass formed during the cultivation process. Thereafter, the HMOs are purified according to the procedures known in the art, e.g., such as described in WO2015188834, WO2017182965 or WO2017152918, and the purified HMOs are used as nutraceuticals, pharmaceuticals, or for any other purpose, e.g., for research.

Manufacturing of HMOs is typically accomplished by performing cultivation in larger volumes. The term “manufacturing” and “manufacturing scale” in the meaning of the invention defines a fermentation with a minimum volume of 5 L culture broth. Usually, a “manufacturing scale” process is defined by being capable of processing large volumes of a preparation containing the product of interest and yielding amounts of the HMO product of interest that meet, e.g., in the case of a therapeutic compound or composition, the demands for clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

With regard to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. Where sucrose is used as the carbon and energy source, a minimal medium might be preferable.

### Method for Producing Specific Blends of LNT and LNFP-I

In an embodiment, the invention relates to a method for the production of a human milk oligosaccharide (HMO) blend with LNFP-I and LNT as the predominant HMO's, the method comprising the steps of, a. providing a genetically engineered cell capable of producing an HMO, b. culturing the cell according to (a) in a suitable cell culture medium to express said nucleic acid sequences and c. harvesting the HMO blend produced in step b, wherein the level of lactose in the culture medium is regulate to modulated the relative levels of LNFP-I and LNT in the produced blend.

In that regard said cell in step a. of the method comprises at least one heterologous β-1,3-N-acetyl-glucosaminyl-transferase protein, LgtA from Neisseria meningitidis as shown in SEQ ID NO: 1 or a functional homologue thereof having an amino acid sequence which is at least 80% identical to SEQ ID NO: 1. Said cell further comprises, at least one heterologous β-1,3-galactosyltransferase protein, GalTK from Helicobacter pylori as shown in SEQ ID NO: 2 or a functional homologue thereof having an amino acid sequence which is at least 80% identical to SEQ ID NO: 2. Said cell additionally comprises at least one heterologous α-1,2-fucosyltransferase protein which is FutC from Helicobacter pylori as shown in SEQ ID NO: 8 or a functional homologue thereof having an amino acid sequence which is at least 80% identical to any one of SEQ ID NO: 8. Said cell also overexpresses the colanic acid gene cluster. In addition, the cell further comprises a native or heterologous regulatory element, optionally in the form of an episomal element, for controlling the expression of heterologous proteins and a native or heterologous regulatory element, optionally in the form of an episomal element, for increasing the expression of native genes and/or proteins. In addition, the cell may also overexpress a gene encoding a native lactose permease protein, which is LacY, as shown in SEQ ID NO: 4, or a functional homologue thereof having an amino acid sequence which is at least 80% identical to SEQ ID NO: 4, and the cell may further comprise a non-functional (or absent) gene product of the DNA-binding transcriptional repressor GlpR, that normally binds to and represses the expression driven by the beforementioned regulatory elements.

As can be seen in example 3, and FIGS. 3 to 7, modulation of the lactose level in the culturing of a cell comprising the genetic modifications mentioned above modulates the level of LNT produced, while the level of LNFP-I remains at about the same level of about 60% to 70% of the total harvested HMO, after 40 hrs of fermentation. In fermentations with low lactose levels, the level of LNT in the harvested blend is reduced to between about 0% to about 15%, while a high lactose level increases the amount of LNT in the produced blend to between about 15% to about 35%.

Accordingly, in embodiments of the method of the present invention, the level of lactose in the culturing media in step b. of the method is modulated. In embodiments the level of lactose during the culturing of the genetically engineered cell in step b. of the method is above 30 g/L, and the molar % of LNT is between 15% and 35%, of the total HMO in the blend produced.

In embodiments the level of lactose during the culturing of the genetically engineered cell in step b. of the method is below 15 g/L, and the molar % of LNT is between 0% and 15%, of the total HMO in the blend produced.

Accordingly, in embodiments, the level of lactose, in the culturing of a cell as described in step b. of the method, is below 15 g/L such as below 10 g/L, such as below 5 g/L or such as below 0.5 g/L, and the ratio of LNFP-I: LNT in the harvested HMOs is in the range of 60:1 to 5:1.

In preferred embodiments, the level of lactose, in the culturing of a cell as described in step b. of the present invention, is above 30 g/L, but below 100 g/L, such as above 40 g/L, such as above 50 g/L or such as above 60 g/L, but below 100 g/L, and the ratio of LNFP-I: LNT in the harvested HMOs is in the range of 4:1 to 2:1.

### Manufactured Product

The term “manufactured product” according to the use of the genetically engineered cell or the nucleic acid construct refer to the one or more HMOs intended as the one or more product HMO(s). The various products are described above.

Advantageously, the methods disclosed herein provides both a decreased ratio of by-product to product and an increased overall yield of the product (and/or HMOs in total). This, less by-product formation in relation to product formation facilitates an elevated product production and increases efficiency of both the production and product recovery process, providing superior manufacturing procedure of HMOs.

The manufactured product may be a powder, a composition, a suspension, or a gel comprising one or more HMOs.

### Tables

TABLE 1
Genotypes of the strains MP1 and MP2

| | | Heterologous Protein |
|---|---|---|
| | | Sequence ID |
| Strain ID | Genotype | (GenBank) |
| MP1 | MDO, x2 GlcNAcT¹, x1 GalTK², x1 CA³, x1 smob⁴ | WP_033911473.1 |
| | | BD182026.1 (modified)⁵ |
| | | WP_126455392.1 |
| MP2 | MDO, x2 GlcNAcT¹, x1 GalTK², x1 CA³, x1 smob⁴, | WP_033911473.1 |
| | x1 PglpF-lacY | BD182026.1 (modified)⁵ |
| | | WP_126455392.1 |

¹GlcNAcT: lgtA gene (SEQ ID NO: 5) coding for the β-1,3-N-acetyloglucosamine transferase LgtA of SEQ ID NO: 1.
²GalTK: gene (SEQ ID NO: 6) coding for the β-1,3-galactosyltransferase GalTK of SEQ ID NO: 2.
³CA: extra colanic acid gene cluster (gmd-wcaG-wcaH-wcaI-manC-manB SEQ ID NO: 45) at a locus that is different than the native locus
⁴smob: (SEQ ID NO: 7) coding for α-1,2-fucosyltransferase of SEQ ID NO: 3
⁵BD182026.1 (modified): compared to BD182026.1, the applied β-1,3-galactosyltransferase sequence has two deletions of 12 and 30 amino acids and shares 90% identity in the homologous regions

TABLE 2
Genotypes of the strains MP3 and MP4

| | | Heterologous Protein |
|---|---|---|
| | | Sequence ID |
| Strain ID | Genotype | (GenBank) |
| MP3 | MDO, x1 GlcNAcT¹, x1 GalTK², x1 CA³, x1 | WP_033911473.1 |
| | Plac(CA)::PglpF-B28⁴, x1 smob⁵ | BD182026.1 (modified)⁶ |
| | | WP_126455392.1 |
| MP4 | MDO, x1 GlcNAcT¹, x1 GalTK², x1 CA³, x1 | WP_033911473.1 |
| | Plac(CA)::PglpF-B28⁴, x1 smob⁵, ΔglpR | BD182026.1 (modified)⁶ |
| | | WP_126455392.1 |

¹GlcNAcT: lgtA gene (SEQ ID NO: 5) coding for the β-1,3-N-acetyloglucosamine transferase LgtA of SEQ ID NO: 1.
²GalTK: gene coding for the β-1,3-galactosyltransferase GalTK of SEQ ID NO: 2.
³CA: extra colanic acid gene cluster (gmd-wcaG-wcaH-wcaI-manC-manB. SEQ ID NO: 45) at a locus that is different than the native locus
⁴Plac(CA): promoter in front of the native CA gene cluster (i.e., Plac) of the MDO platform strain. In strains MP3 and MP4, the Plac is replaced by PglpF_B28.
⁵smob: gene (SEQ ID NO: 7) coding for α-1,2-fucosyltransferase of SEQ ID NO: 3
⁶BD182026.1 (modified): compared to BD182026.1, the applied β-1,3-galactosyltransferase sequence has two deletions of 12 and 30 amino acids and shares 90% identity in the homologous regions

TABLE 3
Genotypes of the strains MP5 and MP6

| | | Heterologous Protein |
|---|---|---|
| | | Sequence ID |
| Strain ID | Genotype | (GenBank) |
| MP5 | MDO, x2 GlcNAcT¹, x1 GalTK², x1 CA³, x1 futC⁴ | WP_033911473.1 |
| | | BD182026.1 (modified)⁵ |
| | | WP_080473865.1 (modified)⁶ |
| MP6 | MDO, x2 GlcNAcT¹, x1 GalTK², x1 CA³, x1 futC⁴, | WP_033911473.1 |
| | | BD182026.1 (modified)⁵ |
| | | WP_080473865.1 (modified)⁶ |

¹GlcNAcT: lgtA gene (SEQ ID NO: 5) coding for β-1,3-N-acetyloglucosamine transferase (SEQ ID NO: 1)
²GalTK: gene (SEQ ID NO: 6)coding for the β-1,3-galactosyltransferase (SEQ ID NO: 2)
³CA: extra colanic acid gene cluster (gmd-wcaG-wcaH-wcaI-manC-manB SEQ ID NO: 45) at a locus that is different than the native locus
⁴futC: gene (SEQ ID NO: 9) coding for α-1,2-fucosyltransferase (SEQ ID NO: 8)
⁵BD182026.1 (modified): compared to BD182026.1, the applied β-1,3-galactosyltransferase sequence has two deletions of 12 and 30 amino acids and shares 90% identity in the homologous regions
⁶WP_080473865.1 (modified): compared to WP_080473865.1, the applied α-1,2-fucosyltransferase has two additional amino acids (LG) at the C-terminus

TABLE 4
HMO blend composition in total broth sample at fermentation timepoint 68.7 h.

| Fermentation | | Process ID | LNFP-I/ | 2′-FL/ | LNT/ | LNT-II/ | |
|---|---|---|---|---|---|---|---|
| Batch ID | | (lactose | HMO | HMO | HMO | HMO | |
| GDF17xxx | Strain | high/low*) | (%) | (%) | (%) | (%) | Remarks |
| 262 | MP5 | L2F20 | 59.9 | 4.0 | 27.2 | 9.0 | Foaming at |
| | | (high) | | | | | 68.7 h |
| 263 | MP5 | L2F21 | 70.0 | 24.7 | 4.0 | 1.3 | Foaming at |
| | | (low) | | | | | 93.0 h |
| 264 | MP6 | L2F20 | 67.5 | 5.9 | 20.7 | 5.9 | Foaming at |
| | | (high) | | | | | 68.7 h |
| 265 | MP6 | L2F21 | 65.7 | 29.5 | 3.4 | 1.1 | No foaming |
| | | (low) | | | | | |

*High/low lactose refers to a concentration range of 30-80 g/L for the high lactose process L2F20 and 0-15 g/L for the low lactose process L2F21, respectively. HMO = sum of HMOs incl. LNFP-I, 2′-FL, LNT, LNT-II, and DFL. All ratios given as molar %.

TABLE 5
Genotypes of the strains MP7, MP8, MP9, MP10 and MP11

| | | Heterologous Protein |
|---|---|---|
| | Genotype | Sequence ID |
| Strain ID | (all genes are genomically integrated) | (GenBank) |
| MP7 | MDO, x2 GlcNAcT¹, x2 GalTK², x1 CA³, x1 smob⁴, | WP_033911473.1 |
| | x1 PglpF-lacY⁸, x1 scrBR⁹, x1 scrYA¹⁰ | BD182026.1 (modified)⁵ |
| | | WP_126455392.1 |
| | | WP_000056853.1 (ScrB) |
| | | WP_000851062.1 (ScrR) |
| | | CAA40657.1 (ScrY) |
| | | CAA40658.1 (ScrA) |
| MP8 | MDO, x2 GlcNAcT¹, x2 GalTK², x1 CA³, x1 smob⁴, | WP_033911473.1 |
| | x1 PglpF-lacY⁸, x1 scrBR⁹, x1 scrYA¹⁰, x1 yberC⁶ | BD182026.1 (modified)⁵ |
| | | WP_126455392.1 |
| | | WP_000056853.1 (ScrB) |
| | | WP_000851062.1 (ScrR) |
| | | CAA40657.1 (ScrY) |
| | | CAA40658.1 (ScrA) |
| | | EEQ08298.1 |
| MP9 | MDO, x2 GlcNAcT¹, x2 GalTK², x1 CA³, x1 smob⁴, | WP_033911473.1 |
| | x1 PglpF-lacY⁸, x1 scrBR⁹, x1 scrYA¹⁰, x1 nec⁷ | BD182026.1 (modified)⁵ |
| | | WP_126455392.1 |
| | | WP_000056853.1 (ScrB) |
| | | WP_000851062.1 (ScrR) |
| | | CAA40657.1 (ScrY) |
| | | CAA40658.1 (ScrA) |
| | | WP_092672081.1 |
| MP10 | MDO, x3 GlcNAcT¹, x2 GalTK², x1 CA³, x1 smob⁴, | WP_033911473.1 |
| | x1 PglpF-lacY⁸, x1 scrBR⁹, x1 scrYA¹⁰, x1 nec⁷ | BD182026.1 (modified)⁵ |
| | | WP_126455392.1 |
| | | WP_000056853.1 (ScrB) |
| | | WP_000851062.1 (ScrR) |
| | | CAA40657.1 (ScrY) |
| | | CAA40658.1 (ScrA) |
| | | WP_092672081.1 |
| MP11 | MDO, x2 GlcNAcT¹, x2 GalTK², x1 CA³, x1 smob⁴, | WP_033911473.1 |
| | x1 PglpF-lacY⁸, x1 scrBR⁹, x1 scrYA¹⁰, x1 yberC⁶, | BD182026.1 (modified)⁵ |
| | x1 nec⁷ | WP_126455392.1 |
| | | WP_000056853.1 (ScrB) |
| | | WP_000851062.1 (ScrR) |
| | | CAA40657.1 (ScrY) |
| | | CAA40658.1 (ScrA) |
| | | EEQ08298.1 |
| | | WP_092672081.1 |

¹GlcNAcT: LgtA gene (SEQ ID NO: 5) coding for the β-1,3-N-acetyloglucosamine transferase (SEQ ID NO: 1) under control of PglpF promoter.
²GalTK: gene (SEQ ID NO: 6) coding for the β-1,3-galactosyltransferase (SEQ ID NO: 2) under control of PglpF promoter.
³CA: extra colanic acid gene cluster (gmd-wcaG-wcaH-wcaI-manC-manB, SEQ ID NO: 45) under control of PglpF promoter inserted at a locus that is different than the native locus
⁴smob: gene (SEQ ID NO: 7) coding for α-1,2-fucosyltransferase (SEQ ID NO: 3) under control of PglpF promoter
⁵BD182026.1 (modified): compared to BD182026.1, the applied β-1,3-galactosyltransferase sequence has two deletions of 12 and 30 amino acids and shares 90% identity in the homologous regions
⁶YberC: MFS transporter with GenBank accession ID EEQ08298.1 under control of Plac promoter
⁷nec: MFS transporter with GenBank accession ID WP_092672081.1 under control of PglpF promoter
⁸LacY: extra copy of native lacY gene inserted at a locus that is different than the native locus
⁹ScrBR: scrBR operon from Salmonella thyphimurium under control of PglpF promoter expressing two genes of a PTS-dependent sucrose utilization transport system encoding SEQ ID NO: 32 and 33
¹⁰ScrYA: scrYA operon from Klebsiella pneumoniae under control of PglpF promoter expressing two genes of a PTS-dependent sucrose utilization transport system encoding SEQ ID NO: 30 and 32

### General

It should be understood that any feature and/or aspect discussed above in connections with the methods according to the invention apply by analogy to the engineered cell, the nucleic acid constructs and/or use described herein.

The terms fermentation and culturing are used interchangeably.

The terms lacto-N-triose, LNT-II, LNT II, LNT2 and LNT 2, are used interchangeably.

The terms genetically engineered and genetically modified are used interchangeably.

The reference to i), ii), iii), iv), v), vi), vii) and viii) throughout the description refers to the components of claim 1.

As shown in Example 3, the fermentation strategies presented herein is effective for producing blends with LNFP-I and LNT as the predominant HMO's, not only with the Smob α-1,2-fucosyltransferase disclosed in SEQ ID NO: 3, but is also for e.g., applying futC as the α-1,2-fucosyltransferase, when the level of lactose is modulated. Thus, it should be understood that any feature and/or aspect discussed above in connections with the Smob α-1,2-fucosyltransferase according to the disclosure apply by analogy to the futC α-1,2-fucosyltransferase in conjunction with modulation of the level of lactose as disclosed in Example 3. In the present context, a heterologous α-1,2-Fucosyltransferase futC protein is as shown in SEQ ID NO: 8 or a functional homologue thereof having an amino acid sequence which is at least 80% identical to any one of SEQ ID NO: 8.

The following figures and examples are provided below to illustrate the present disclosure. They are intended to be illustrative and are not to be construed as limiting in any way.

### BRIEF DESCRIPTION OF THE FIGURES

FIG. 1

The effect of lacY expression levels on the HMO content (in % mM) of the final blends generated by strains (a) MP1 and (b) MP2.

FIG. 2

The effect of glpR+/−phenotype on the HMO content (in % mM) of the final blends generated by strains (a) MP3 and (b) MP4.

FIG. 3

Time profiles for the lactose monohydrate concentration in the fermentation broth throughout the four runs at either high lactose (process L2F20) or low lactose (process L2F21) condition using the two strains MP5 and MP6.

FIG. 4

Time profiles of the LNFP-I/HMO ratio in the fermentation broth throughout the four runs at either high lactose (process L2F20) or low lactose (process L2F21) condition using the two strains MP5 and MP6. HMO=sum of HMOs incl. LNFP-I, 2′-FL, LNT, LNT-II and DFL. DFL is <0.3 g/L.

FIG. 5

Time profiles of the molar ratio 2′-FL/HMO in the fermentation broth throughout the four runs at either high lactose (process L2F20) or low lactose (process L2F21) condition using the two strains MP5 and MP6. HMO=sum of HMOs incl. LNFP-I, 2′-FL, LNT, LNT-II and DFL. DFL is <0.3 g/L.

FIG. 6

Time profiles of the molar ratio LNT/HMO in the fermentation broth throughout the four runs at either high lactose (process L2F20) or low lactose (process L2F21) condition using the two strains MP5 and MP6. HMO=sum of HMOs incl. LNFP-I, 2′-FL, LNT, LNT-II and DFL. DFL is <0.3 g/L.

FIG. 7

Time profiles of the molar ratio LNT-II/HMO in the fermentation broth throughout the four runs at either high lactose (process L2F20) or low lactose (process L2F21) condition using the two strains MP5 and MP6. HMO=sum of HMOs incl. LNFP-I, 2′-FL, LNT, LNT-II and DFL. DFL is <0.3 g/L.

FIG. 8

LNFP-I and LNT titers reached in cell cultures containing sucrose as the carbon source for smob-expressing strains that bear a genomic copy of the yberC gene (strain MP8) or the nec gene (strain MP9) or both (strain 11) relative to cells that do not express an MFS transporter (strain MP7). The relative HMO titers of a strain (MP10) bearing both a nec-expression cassette and an extra IgtA-expression cassette relative to HMO in strain MP7 is also depicted in the figure. Titers are shown in % relative to total HMO of each individual strain.

### SEQUENCE ID'S

The current application contains a sequence listing in text format and electronical format which is hereby incorporated by reference as are the sequences listed in the corrected sequence list in the priority application DK PA 2021 70251. Below is a summary of the sequences included in the application.
SEQ ID NO: 1 [Lgta protein]
SEQ ID NO: 2 [GalTK protein]
SEQ ID NO: 3 [Smob protein]
SEQ ID NO: 4 [LacY protein]
SEQ ID NO: 5 [Igta gene]
SEQ ID NO: 6 [galTK gene]
SEQ ID NO: 7 [smob gene]
SEQ ID NO: 8 [futC protein]
SEQ ID NO: 9 [futC gene]
SEQ ID NO: 10 [lacY gene]
SEQ ID NO: 11 [Pscr]
SEQ ID NO: 12 [pgatY_70UTR]
SEQ ID NO: 13 [PglpF]
SEQ ID NO: 14 [PglpF_SD1]
SEQ ID NO: 15 [PglpF_SD10]
SEQ ID NO: 16 [PglpF_SD2]
SEQ ID NO: 17 [PglpF_SD3]
SEQ ID NO: 18 [PglpF_SD4]
SEQ ID NO: 19 [PglpF_SD5]
SEQ ID NO: 20 [PglpF_SD6]
SEQ ID NO: 21 [PglpF_SD7]
SEQ ID NO: 22 [PglpF_SD8]
SEQ ID NO: 23 [PglpF_SD9]
SEQ ID NO: 24 [PglpF_B28]
SEQ ID NO: 25 [PglpF_B29]
SEQ ID NO: 26 [Plac_16UTR]
SEQ ID NO: 27 [Plac]
SEQ ID NO: 28 [PmglB_70UTR]
SEQ ID NO: 29 [PmglB_70UTR_SD4]
SEQ ID NO: 30 [scrY]
SEQ ID NO: 31 [scrA]
SEQ ID NO: 32 [scrB]
SEQ ID NO: 33 [scrR]
SEQ ID NO: 34 [SacC_Agal]
SEQ ID NO: 35 [Bff]
SEQ ID NO: 36 [Pscr_SD1]
SEQ ID NO: 37 [Pscr_SD7]
SEQ ID NO: 38 [Bad]
SEQ ID NO: 39 [Nec]
SEQ ID NO: 40 [YberC]
SEQ ID NO: 41 [Fred]
SEQ ID NO: 42 [Vag]
SEQ ID NO: 43 [Marc]
SEQ ID NO: 44 [glpR gene]
SEQ ID NO: 45 [colanic acid gene cluster]

### EXAMPLES

### Example 1—Generation of Variations of HMO Blends of LNFP-I, 2′-FL and LNT by Increasing the Expression of the Gene Coding Lactose Permease

### Description of the Genotype of Strains MP1 and MP2 Tested in Deep Well Assays

As background strains for the strains the bacterial strain MDO, was used. MDO is constructed from Escherichia coli K-12 DH1. The E. coli K-12 DH1 genotype is: F⁻, λ−, gyrA96, recA1, relA1, endA1, thi-1, hsdR17, supE44. In addition to the E. coli K-12 DH1 genotype MDO has the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene (WO2019/123324A1).

Based on the platform strain (“MDO”), the modifications summarised in table 1, were made to obtain the LNFP-I producing strains MP1 and MP2 used in this study, both being fully chromosomal strains. The strains can produce the tetrasaccharide HMO LNT and fucosylate LNT further to obtain the pentasaccharide HMO, LNFP-I.

The fucosyltransferase enzyme used for this reaction, namely the Smob enzyme (α-1,2-fucosyl-transferase), derived from Sulfuriflexus mobilis, was found to be able to fucosylate LNT with an extraordinary specificity to yield LNFP-I as the predominant product in the final HMO blend. Likewise, other HMOs, such as 2′-FL, LNT and LNT-II are present in the final HMO blend, but at much lower concentrations.

In the present Example, it was demonstrated how the over-expression of the lacY gene coding the lactose permease LacY is used as a genetic tool to obtain a specific target composition of a HMO mixture comprising up to four HMOs, including LNFP-I, LNT, 2′-FL and LNT-II (in order of abundance).

This invention demonstrates how the over-expression of the lacY gene can be advantageously used to modulate the composition of the HMO blend produced by strains MP1 and MP2. The only difference between the two strains, as shown in the table below, is the knock-out of a locus encoding a non-essential gene by insertion of the PglpF-lacY expression cassette at this locus. The gene product of lacY is the lactose permease LacY that enables the import of lactose from the cell exterior into the cytoplasm. Over-expression of the lacY gene is therefore believed to enhance lactose import into the cell and thereby HMO production in multiple manners.

### Description of the Applied Deep Well Assay Protocol for Strain Characterization

The strains disclosed in the present example were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium supplemented with magnesium sulphate, thiamine and glucose. The precultures were incubated for 24 hours at 34° C. and 1000 rpm shaking and then further transferred to a new basal minimal medium (BMM, pH 7.5) in order to start the main culture. The new BMM was supplemented with magnesium sulphate, thiamine, a bolus of 20% glucose solution (50 μl per 100 mL) and a bolus of 10% lactose solution (5 ml per 100 ml). Moreover, 50% sucrose solution was provided as carbon source, accompanied by the addition of sucrose hydrolase (invertase), so that glucose was released at a rate suitable for C-limited growth. The main cultures were incubated for 72 hours at 28° C. and 1000 rpm shaking.

For the analysis of total broth, the 96-well plates were boiled at 100° C., subsequently centrifuged, and finally the supernatants were analysed by HPLC. For supernatant samples, the initial centrifugation of microtiter plates was followed by the removal of 0.1 mL supernatant for direct analysis by HPLC. For pellet samples, the cells were initially washed, then dissolved in deionized water and centrifuged. Following centrifugation, the pellets were analysed for HMO content in the cell interior after resuspension, boiling, centrifugation and analysis of the final supernatant.

### Results of the Deep Well Assays

Strains were characterized in deep well assays and total samples were analysed for HMO content by HPLC following the 72-hour protocol described above. The millimolar content (mM) of the detected HMOs in each sample was calculated based on the reported analytical data, and the mM percentage (%) of each HMO in the final blend was calculated in order to easily compare the quantitative differences in the HMO blends generated by each strain.

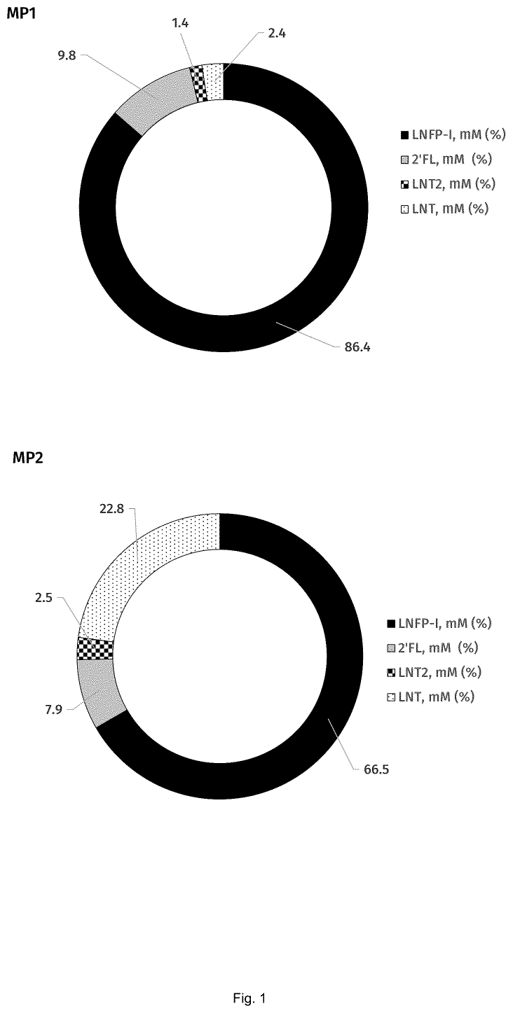

As shown in FIG. 1, a single genetic modification, namely the over-expression of the lacY gene, resulted in a drastic change in the acquired HMO blend. The increase of lacY expression levels clearly reduces the formation of LNFP-I and 2′-FL and markedly increases the formation of LNT and LNT-II. Specifically, the strain with wild-type expression levels of the lacY gene, MP1, generates a blend consisting of 86% LNFP-I and 10% 2′-FL, while the strain over-expressing lacY, MP2, provides a blend with 20% less LNFP-I and 20% more LNT compared to MP1.

It is noteworthy that the total HMO concentration in the corresponding final HMO blends generated by the strains MP1 and MP2 are the same (data not shown).

In conclusion, the over-expression of the lacY gene changed the relevant HMO abundance in such a manner that the final blend consists mainly of LNFP-I and LNT (MP2) instead of LNFP-I and 2′-FL (MP1).

### Example 2-Generation of Variations of HMO Blends of LNFP-I, 2′-FL and LNT by Deleting the glpR Gene that Represses PglpF-Driven Gene Expression

### Description of the Genotype of Strains MP3 and MP4 Tested in Deep Well Assays

Based on the platform strain (“MDO”) described in example 1, the modifications summarised in table 2, were made to obtain the LNFP-I producing strains MP3 and MP4 used in this study, both being fully chromosomal strains. The strains are capable of producing the tetrasaccharide HMO LNT and fucosylate LNT further to obtain the pentasaccharide HMO LNFP-I. The fucosyltransferase enzyme used for this reaction, namely the Smob enzyme (α-1,2-fucosyl-transferase), derived from Sulfuriflexus mobilis, was found to be able to fucosylate LNT with an extraordinary specificity to yield LNFP-I as the predominant product in the final HMO blend. Likewise, other HMOs, such as 2′-FL, LNT and LNT-II are present in the final HMO blend, but at much lower concentrations. In the present Example, it was demonstrated how the deletion of the glpR gene coding the DNA-binding transcriptional repressor GlpR is used as a genetic tool to obtain a specific target composition of a HMO mixture comprising up to four HMOs, including LNFP-I, LNT, LNT-II and 2′-FL (in order of abundance).

This invention demonstrates how the deletion of the glpR gene can be advantageously used to modulate the composition of the HMO blend produced by strains MP3 and MP4. The only difference between the two strains, as shown in the table below, is the knock-out of the glpR gene. The gene product of glpR is the DNA-binding transcriptional repressor GlpR, which acts as the repressor of the glycerol-3-phosphate regulon, which is organized in different operons. One of its targets is the PglpF promoter, which is originally found in front of the native E. coli gene glpF, which codes the glycerol facilitator GlpF. Since the colanic acid gene cluster and the heterologous genes coding glycosyltransferases for HMO synthesis are under the control of the PglpF promoter, the deletion of the glpR gene eliminates the GlpR-imposed repression of transcription from all PglpF promoters in the cell and in this manner it can enhance gene expression from all PglpF-based cassettes that are present in the genome of the host.

### Description of the Applied Deep Well Assay Protocol for Strain Characterization

The strains disclosed in the present example were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium supplemented with magnesium sulphate, thiamine and glucose. The precultures were incubated for 24 hours at 34° C. and 1000 rpm shaking and then further transferred to a new basal minimal medium (BMM, pH 7.5) in order to start the main culture. The new BMM was supplemented with magnesium sulphate, thiamine, a bolus of 20% glucose solution (50 μl per 100 mL) and a bolus of 10% lactose solution (5 ml per 100 ml). Moreover, 50% sucrose solution was provided as carbon source, accompanied by the addition of sucrose hydrolase (invertase), so that glucose was released at a rate suitable for C-limited growth. The main cultures were incubated for 72 hours at 28° C. and 1000 rpm shaking.

For the analysis of total broth, the 96-well plates were boiled at 100° C., subsequently centrifuged, and finally the supernatants were analysed by HPLC. For supernatant samples, the initial centrifugation of microtiter plates was followed by the removal of 0.1 mL supernatant for direct analysis by HPLC. For pellet samples, the cells were initially washed, then dissolved in deionized water and centrifuged. Following centrifugation, the pellets were analysed for HMO content in the cell interior after resuspension, boiling, centrifugation and analysis of the final supernatant.

### Results of the Deep Well Assays

Strains were characterized in deep well assays and total samples were analysed for HMO content by HPLC following the 72-hour protocol described above. The millimolar content (mM) of the detected HMOs in each sample was calculated based on the reported analytical data, and the mM percentage (%) of each HMO in the final blend was calculated in order to easily compare the quantitative differences in the HMO profiles acquired by each strain.

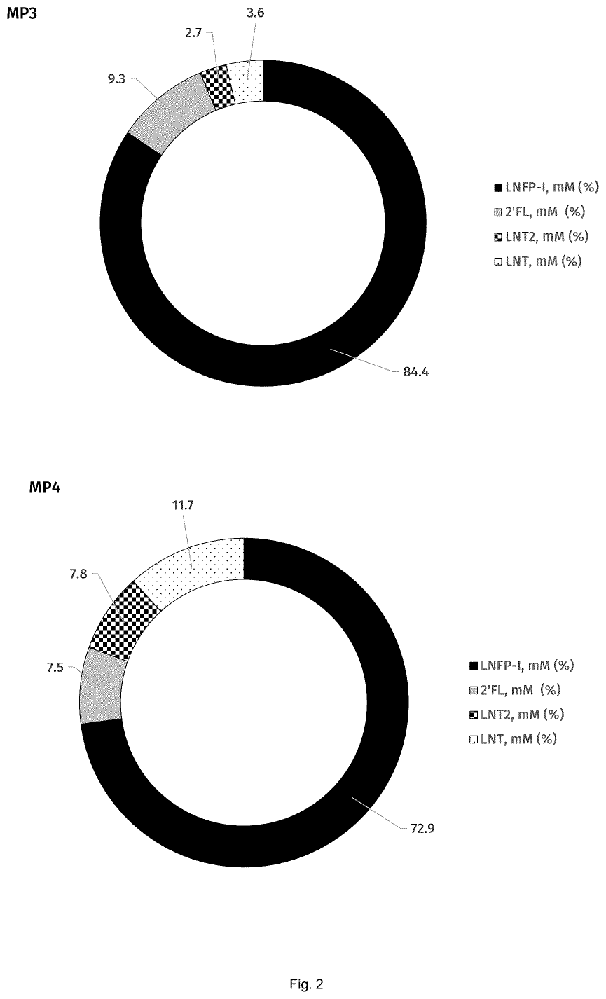

As shown in FIG. 2, a single genetic modification, namely the deletion of the glpR gene, resulted in a notable change in the acquired HMO blend. Deletion of the glpR gene reduced the formation of LNFP-I and increased the formation of LNT and LNT-II to a certain extend (approximately 10% changes in the abundance of each of these HMOs). Specifically, the glpR+strain, MP3, generated a blend consisting of 84% LNFP-I and 10% 2′-FL, while the glpR-strain, MP4, provided a blend with 10% less LNFP-I and 8% more LNT compared to MP3.

It is noteworthy that the deletion of the glpR gene resulted in approximately 10% higher total HMO concentration in the blend acquired by the strain MP4 compared to the one generated by the strain MP3 (data not shown).

In conclusion, the deletion of the glpR gene changed the relevant HMO abundance in such a manner that the abundance of LNFP-I and 2′FL was decreased by 11% and 2%, respectively, while an increase in LNT by 8% and LNT2 by 5% was observed.

### Example 3—Generation of Variations of HMO Blends of LNFP-I, 2′-FL and LNT by Lactose Concentration Modulation During Fermentation

Description of the Genotype of Strains MP5 and MP6 Tested in Fermentations with High or Low Lactose Process

Based on the platform strain (“MDO”) described in example 1, the modifications summarised in the Table 3, were made to obtain the LNFP-I producing strains MP5 and MP6 used in this study, both being fully chromosomal strains. The strains are capable of producing the tetrasaccharide HMO LNT and fucosylate LNT further to obtain the pentasaccharide HMO LNFP-I. The fucosyltransferase enzyme used for this reaction, namely the FutC enzyme (α-1,2-fucosyltransferase), derived from Helicobacter pylori (GenBank ID: WP_080473865.1, but with two additional amino acids (LG) at the C-terminus), was found to be able to fucosylate LNT to yield LNFP-I as predominant product of these strains. Likewise, other HMOs are being produced, with 2′-FL, LNT and LNT-II being the predominant side products at varying concentrations, depending on the growth conditions in fermentation, in particular the concentration of the acceptor lactose during fermentation.

In the present Example, it is demonstrated how modulation of the lactose level during fermentation is used to obtain a specific target composition of a HMO mixture comprising up to four HMOs in significant quantities of LNFP-I, 2′-FL, LNT and LNT-II. Therefore, this invention deals with how lactose addition during fermentation can be advantageously used to modulate the composition of the HMO blend produced by strains MP5 and MP6. The only difference between the two strains lies in genomic loci that were selected for the integration of the heterologous glycosyltransferases

Description of the Fermentation Processes with High and Low Lactose Levels

The fermentations were carried out in 200 mL DasBox bioreactors (Eppendorf, Germany), starting with 100 ml of defined mineral culture medium, consisting of 30 g/kg carbon source (glucose), MgSO4×7H2O, KOH, NaOH, NH4H2PO4, KH2PO4, trace element solution, citric acid, antifoam and thiamine. The trace metal solution (TMS) contained Mn, Cu, Fe, Zn as sulfate salts and citric acid. Fermentations were started by inoculation with 2% (v/v) of pre-cultures grown in a defined minimal medium. After depletion of the carbon source contained in the batch medium, a sterile feed solution containing glucose, MgSO4×7H2O, TMS and H3PO4 was fed continuously in a carbon-limited manner using a predetermined, linear profile.

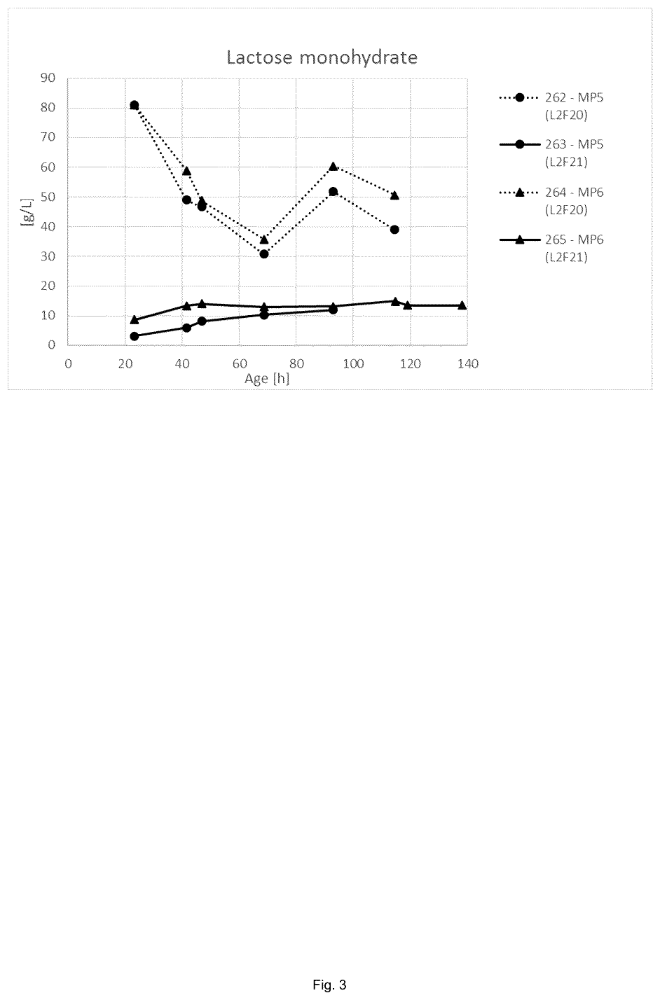

Lactose addition was done in two different ways, depending on if a high or low lactose process was chosen. In the high lactose process (“L2F20”), lactose monohydrate solution was added by two bolus additions, the first one at approx. 10 hours after feed start, the second one at approx. 70 hours EFT. In the low lactose process (“L2F21”), lactose was fed continuously as part of the glucose feed solution, with no bolus additions. As shown in FIG. 3, this resulted in the following lactose concentration ranges: high lactose process 30-80 g/L, low lactose process 0-15 g/L.

The pH throughout fermentation was controlled at 6.8 by titration with 14% NH₄OH solution. Aeration was controlled at 1 vvm using air, and dissolved oxygen was kept above 23% of air saturation, controlled by the stirrer rate. At 15 min after glucose feed start, the fermentation temperature setpoint was lowered from 33° C. to 25° C. This temperature drop was conducted instantly without a ramp. Fermentations were operated either until instability in terms of excessive foaming was observed, or until a maximum duration of almost 140 hours, as specified in Table 4.

Throughout the fermentations, samples were taken in order to determine the concentration of LNFP-I, 2′-FL, LNT, LNT-II, DFL, lactose and other minor by-products using HPLC. Total broth samples were diluted three-fold in deionized water and boiled for 20 minutes. This was followed by centrifugation at 17000 g for 3 minutes, where after the resulting supernatant was analysed by HPLC. The above measurements were used to accurately calculate the ratios of each HMO relative to the total sum of HMO (“HMO”), comprising LNFP-I, 2′-FL, LNT, LNT-II and DFL.

### Results of the Fermentation Runs

The four fermentations ran in a stable manner for at least 68.7 h. In three instances, excessive foaming occurred late in fermentation, while GDF17265 ran in a very stable manner for 138.3 hours. For the reason of comparison, Table 4 depicts HMO compositions in fermentation samples at timepoint 68.7 h. The numbers represent ratios of the individual HMOs LNFP-I, 2′-FL, LNT and LNT-II as a ratio to the total sum of these four HMOs including DFL (“HMO”), in molar-%. DFL numbers are not shown since this HMO only appears in traces of up to 0.3 g/L. As depicted in FIG. 3, the two processes allow to maintain lactose levels of 30-80 g/L for the high lactose process L2F20 and 0-15 g/L for the low lactose process L2F21. The two fermentation processes are otherwise identical with regard to medium composition, glucose feed profile and fermentation process parameters such as temperature, pH and dissolved oxygen.

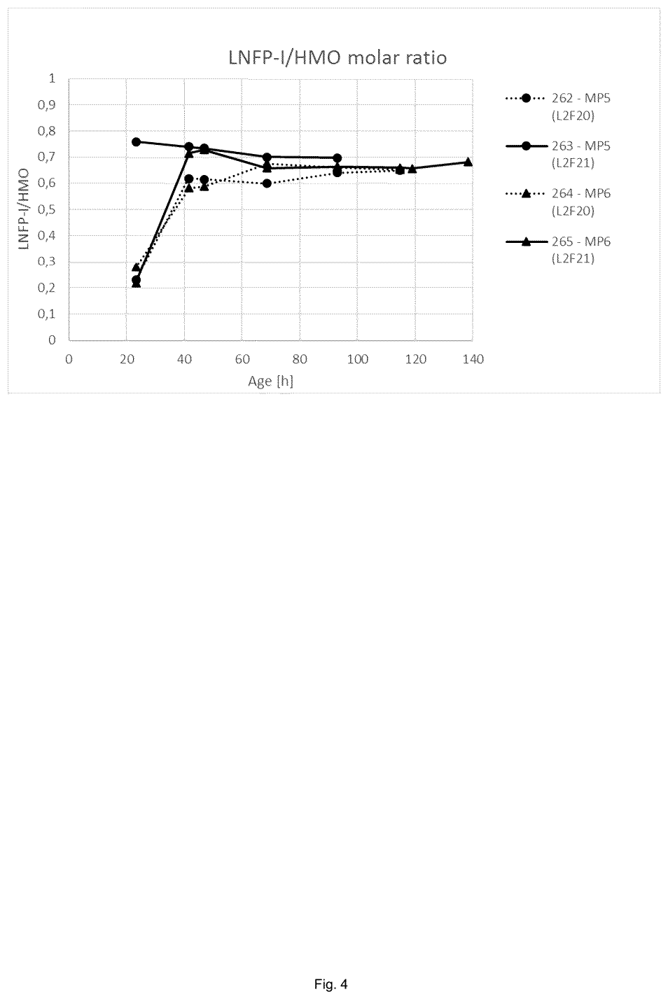

Furthermore, as depicted in FIG. 4, the predominant HMO product in all fermentations is LNFP-I in a range between approximately 60% and 70% of the total HMO produced in molar %. This level is already reached early in fermentation, i.e., from approximately 40 hours, and remains almost unchanged throughout. Moreover, the LNFP-I/HMO ratio is almost independent from lactose levels, i.e., only slightly higher with the low lactose process for both strains tested.

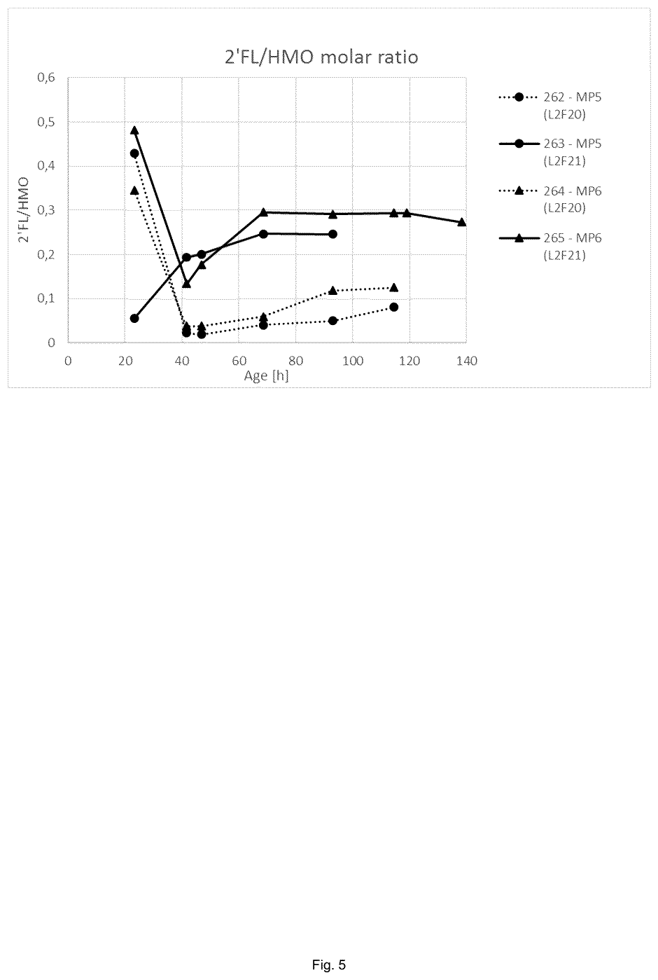

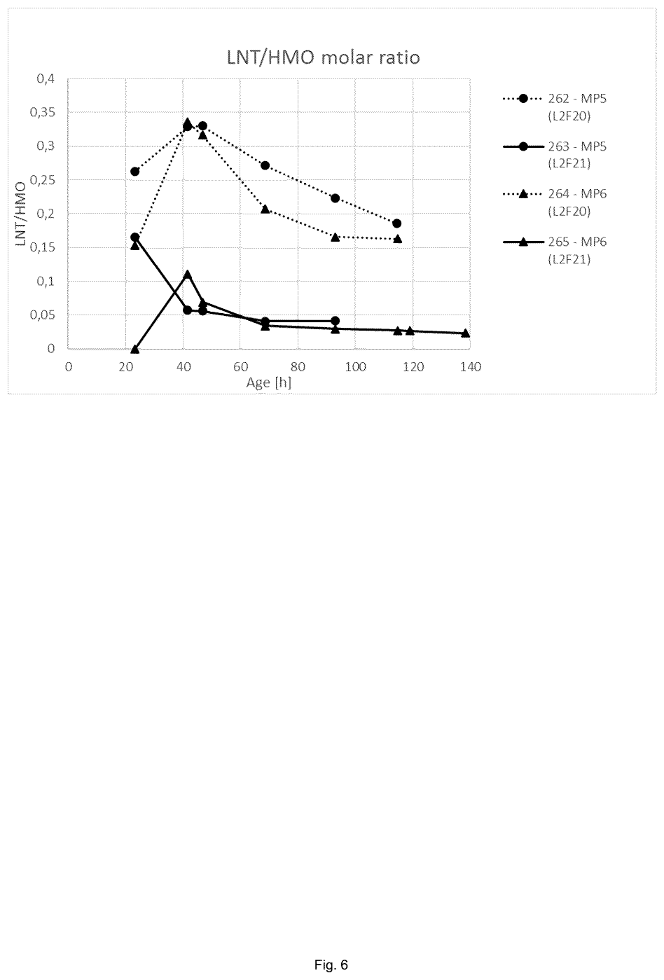

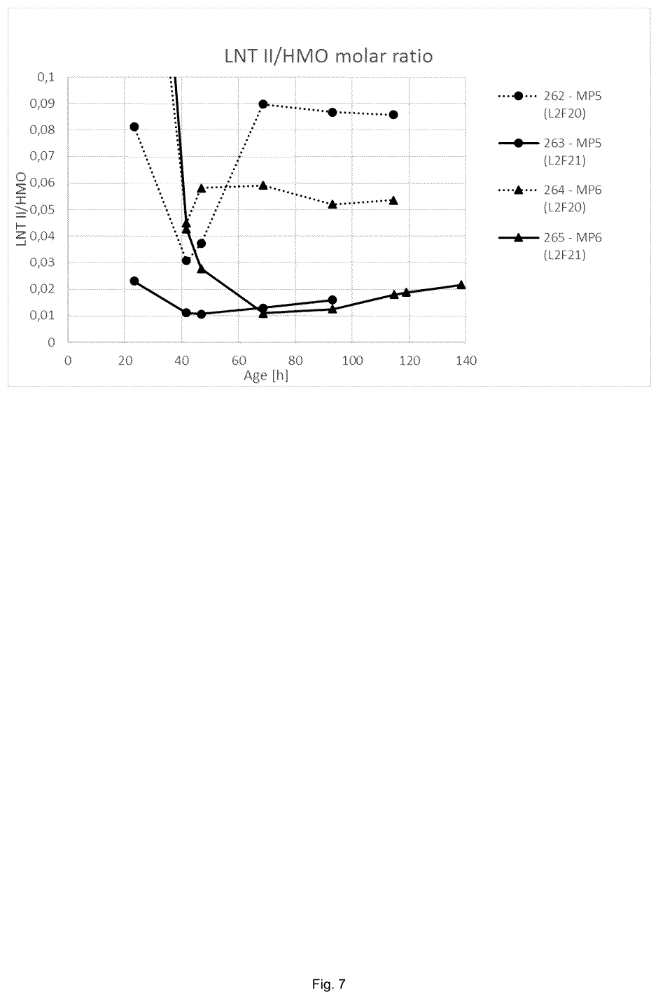

Finally, FIGS. 5, 6 and 7 depict the time profiles of the three product ratios 2′-FL/HMO, LNT/HMO and LNT-II/HMO in the fermentation broth throughout the four runs. The data reveal two major product compositions which are highly dependent on the lactose concentration. In the high lactose process (L2F20), the HMO product profile is (in decreasing order) LNFP-I>2′-FL>LNT>LNT-II. In the low lactose process (L2F21), the HMO product profile is LNFP-I>LNT>LNT-II>=2′-FL. This applies equally to both strains investigated. Thus, as shown in Table 4, the following ranges can be found for the high lactose process in molar %: LNFP-I/HMO [60-68], LNT/HMO [21-27], LNT-II/HMO [6-9], 2′-FLHMO [4-6]. And the following ranges were found for the low lactose process in xmolar %: LNFP-I/HMO [66-70], 2′-FL/HMO [25-30], LNT/HMO [3-4], LNT-II/HMO 1-1.5]. Hence, lactose concentration can be powerful control tool to achieve a pre-determined, desired profile of 3-4 major HMOs during the production of HMO blends containing predominantly LNFP-I.

### Example 4-Generation of Variations of HMO Blends of LNFP-I and LNT by Increasing the Expression of a β-1,3 GlcNAc Transferase and/or Introducing Heterologous Genes Encoding Transporters of the Major Facilitator Superfamily (MFS)

### Description of the Genotype of Strains MP7, MP8, MP9, MP10 and MP11 Tested in Deep Well Assays

Based on the platform strain (“MDO”) described in example 1, the modifications summarised in Table 5, were made to obtain the fully chromosomal strains MP7, MP8, MP9, MP10 and MP11. The strains can produce the pentasaccharide HMO LNFP-I and the tetrasaccharide HMO LNT. The glycosyltransferase enzymes LgtA (a β-1,3-N-acetyloglucosamine transferase, SEQ ID NO: 1) from N. meningitidis, GalTK (a β-1,3-galactosyltransferase, SEQ ID NO: 2) from H. pylori and Smob (α-1,2-fucosyltransferase, SEQ ID NO: 3) from S. mobilis are present in all five strains. Furthermore, all strains possess an extra copy of the native E. coli gene lacY (SEQ ID NO: 10), which encodes lactose permease. Strains MP7-MP11 are all able to utilize sucrose as the carbon and energy source since the operons scrBR from Salmonella thyphimurium plasmid pUR400 and scrYA from Klebsiella pneumoniae are integrated on their genome. Moreover, the strain MP8 expresses the heterologous transporter of the Major Facilitator Superfamily (MFS) YberC from Yersinia bercovieri, while the strains MP9 and MP10 express the heterologous MFS transporter Nec from Rosenbergiella nectarea. In addition, the strain MP10 has three PglpF-driven copies of the IgtA gene, while the rest strains have only two. Finally, the strain MP11 bears one copy of each of the above-mentioned transporter-encoding genes. The strength of the promoter that drives the expression of the yberC and nec genes differs, i.e., a PglpF-driven nec copy is present in the strains MP9, MP10 and MP11, while the strains MP8 and MP11 expresses the yberC gene under the control of the Plac promoter.

This invention demonstrates how the introduction of heterologous MFS transporters, potentially combined with an increase in the expression levels of a selected β-1,3 GlcNAc transferase can be advantageously used to modulate the composition of the HMO blend produced by strain MP7. The only difference between the strains MP7 and MP8-MP11, as shown in the table below, is the copy number of the β-1,3 GlcNAc transferase converting lactose to LNT-II and/or the presence of MFS-encoding expression cassette(s) that were integrated on the genome of the strains on top of the pre-existing modifications in strain MP7.

Introduction of relevant expression cassettes for the yberC and/or nec genes on the genome of a highly producing LNFP-I/LNT producing cell is expected to modulate the balance of available precursor and final sugar products both in the cell interior and its exterior. The introduction of MFS-encoding cassettes is therefore a perfect genetic tool to adjust in multiple manners the HMO profile that results at the end of a fermentation process. On the other hand, increasing the expression of a given β-1,3 GlcNAc transferase can be beneficial for the increase of the LNT and/or the LNFP-I titer, thus favouring the prevalence of either LNT or LNFP-I in the final HMO profile (blend).

### Description of the Applied Deep Well Assay Protocol for Strain Characterization

The strains disclosed in the present example were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium (BMM) supplemented with magnesium sulphate, thiamine and glucose. The precultures were incubated for 24 hours at 34° C. and 1000 rpm shaking and then further transferred to a new BMM (pH 7.5) to start the main culture. The new BMM was supplemented with magnesium sulphate, thiamine, a bolus of 20% glucose solution (0.5 μL per mL) and a bolus of 20% lactose solution (0.1 μL per μL). Moreover, a 20% stock solution of a specific polysaccharide was provided as carbon source, accompanied by the addition of a specific hydrolytic enzyme, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analysed by HPLC. The samples were made with three or four replicates.

### Results of the Deep Well Assays

The present example investigates further genetic tools to adjust HMO ratios at the levels desired by a customer or a specific biological study that will use the final material generated by a fermentation process involving the genetically engineered cell.

The identification of sugar exporters and the fine balancing of their expression can be a key for the success of HMO production systems. This task can though be challenging, since only the HMO of interest, and not the precursor or elongated versions thereof, should be bound and exported by the chosen sugar exporter. No matter the manner that the final HMO profile is modulated by the introduction of one or more sugar transporters in the HMO-producing cell, transporter proteins constitute an ideal genetic tool for acquiring desired HMO profiles at the end of a fermentation process.

Sugar transporters proven to be able to export mainly the LNT and to a lesser extent the LNFP-I product to the cell exterior, namely Nec and YberC, were introduced in the LNFP-I sucrose strain MP7. Relevant strains were constructed and characterized in deep well assays as described in the previous sections. Samples were collected from the total broth of the cultures. All samples were analysed for HMO content by HPLC following the 72-hour protocol described above. The concentration of the detected HMOs (titer) in each sample was used to calculate the molar % of different based on the total HMO content of the strain tested

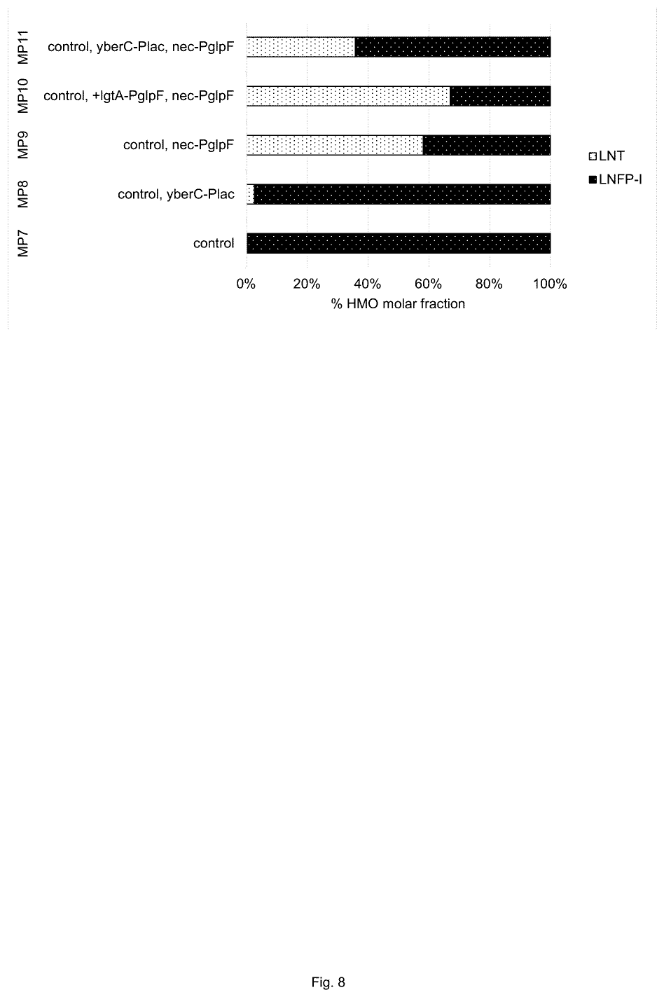

As revealed by the analysis of the total samples in deep-well cultures, the introduction of a Plac-driven copy of the gene encoding the YberC MFS transporter in the genetic background of the strain MP7 to create strain MP8 increase the LNT titer slightly (FIG. 8), while the LNFP-I titer remains at the original levels of MP7. A single PglpF-driven copy of the gene encoding the Nec MFS transporter in the genetic background of the strain MP7 to create strain MP9, with more than doubles the final LNT titer of the strain MP9 and markedly reduces its LNFP-I titer to half compared to the strain MP7 (FIG. 8). Furthermore, the combination of two modifications, namely the further increase of IgtA expression and the introduction of a nec-containing cassette in the genetic background of MP7 to create the strain MP10 enhances the above-mentioned HMO profile observed for the strain MP9, i.e., the LNT titer reaches very high levels (higher than with strain MP9, where the nec gene alone was introduced in the strain MP7) and the LNFP-I titer drops even further (FIG. 8).

Given these above results with MFS-containing constructs, it was interesting to combine both the yberC- and the nec-constructs in a single strain and note how the LNFP-I and LNT titers were affected. Introduction of both constructs in the genetic background of the strain MP7 to create the strain MP11 retained ¾ of its original LNFP-I titer, while it resulted in the formation of much higher LNT titers compared to the ones observed for the strain MP7 and MP8. Notably though the LNT titer of the strain MP11 is lower than the one observed for the strain MP9.

In conclusion, the genetic tools presented here for the modulation of LNFP-I and LNT titers obtained by a sucrose-utilizing, high-producing strain are very powerful towards the construction of customized LNFP-V/LNT profiles (blends). As shown above, the production of LNT can be largely favored by the introduction of a nec- and/or an extra IgtA-containing cassette. On the contrary, if it is desired to have LNFP-I as the most prevalent HMO in the final HMO blend acquired by a modified cell, then the yberC-containing cassette should be introduced in the cell together with the nec cassette.

1. A method for the production of a human milk oligosaccharide (HMO) blend with LNFP-I and LNT as the predominant HMO's, the method comprising the steps of
a) providing a genetically engineered cell capable of producing an HMO, wherein said cell expresses
i) a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
ii) a heterologous β-1,3-galactosyltransferase protein as shown in SEQ ID NO: 2 or a functional homologue thereof having an amino acid sequence which is at least 80% identical to SEQ ID NO: 2; and
iii) a heterologous α-1,2-fucosyltransferase protein as shown in SEQ ID NO: 3 or 8 or a functional homologue thereof having an amino acid sequence which is at least 80% identical to any one of SEQ ID NO: 3 or 8, and
iv) a lactose permease protein as shown in SEQ ID NO: 4, or a functional homologue thereof having an amino acid sequence which is at least 80% identical to SEQ ID NO: 4, and
v) a functional colanic acid gene cluster,
and further comprises
vi) a native or heterologous regulatory element for controlling the expression of i) ii) iii) and v), and
vii) a native or heterologous regulatory element for increasing the expression of iv) and/or
viii) a non-functional or absent gene product that normally binds to and represses the expression driven by vi)-vii)
b) culturing the cell according to (a) in a suitable cell culture medium to produce said HMO blend; and
c) harvesting the HMO blend produced in step (b).

2. The method according to claim 1, wherein the heterologous α-1,2-fucosyltransferase in iii) corresponds to SEQ ID NO: 3 or a functional homologue thereof having an amino acid sequence which is at least 80% identical to any one of SEQ ID NO: 3.

3. The method according to claim 1, wherein an over-expression of any of the protein(s) in i)-iv) is provided by increasing the copy number of the genes coding said protein(s).

4. The method according to claim 1, wherein controlling the expression of the colanic acid gene cluster is modulated by swapping the native promoter with a promoter of interest, and/or increasing the copy number of the colanic acid genes coding said protein(s), or episomally expressing the colanic acid gene cluster or expressing it from a different locus on the chromosome.

5. The method according to claim 1, wherein the regulatory element for controlling and increasing the expression of i)-v) is a promoter selected from any one of SEQ ID NO: 11 to 29.

6.-7. (canceled)

8. The method according to claim 1, wherein the gene product in vii) is the transcriptional repressor GlpR.

9. The method according to claim 1, wherein the cell further comprises a recombinant nucleic acid sequence encoding the sugar transport protein(s) YberC and/or Nec.

10. The method according to claim 1, wherein the level of lactose the fermentation medium during the culturing of the genetically engineered cell in step (b) is between 30 to 80 g/L.

11. The method according to according to claim 1, wherein LNFP-I and LNT are the predominant HMOs with a molar % of LNT and LNFP-I combined is above 75% of the total HMO.

12. The method according to claim 1, wherein the HMO blend has a molar % of LNT between 10% to 70% and LNFP-I between 30% to 95% of the total HMO.

13. The method according to claim 1, wherein the ratio of LNFP-I: LNT in the harvested HMOs is 10:1, 5:1, 3:1, 5:2, 2:3 or 1:3.

14. A genetically engineered cell comprising
d) one or more nucleic acid sequence(s) encoding a heterologous β-1,3-N-acetyl-glucosaminyl-transferase protein as shown in SEQ ID NO: 1 or a functional homologue thereof having an amino acid sequence which is at least 80% identical to SEQ ID NO: 1; and
e) one or more nucleic acid sequence(s) encoding a heterologous β-1,3-galactosyltransferase protein as shown in SEQ ID NO: 2 or a functional homologue thereof having an amino acid sequence which is at least 80% identical to SEQ ID NO: 2; and
f) one or more nucleic acid sequence(s) encoding a heterologous α-1,2-fucosyltransferase protein as shown in SEQ ID NO: 3 or a functional homologue thereof having an amino acid sequence which is at least 80% identical to any one of SEQ ID NO: 3, and
g) one or more nucleic acid sequence(s) encoding one or more nucleic acid sequence(s) encoding a lactose permease protein as shown in SEQ ID NO: 4, or a functional homologue thereof having an amino acid sequence which is at least 80% identical to SEQ ID NO: 4, and
h) more than one nucleic acid sequence(s) encoding the proteins of the colanic acid gene cluster.

15. The genetically engineered cell according to claim 14 further comprising
i) a native or heterologous regulatory element for controlling the expression of a), b) or c), and
ii) a native or heterologous regulatory element for increasing the expression of d), and/or
iii) a non-functional or absent gene product that normally binds to and represses the expression driven i) and/or ii).

16. The genetically engineered cell according to claim 14, wherein the lactose permease protein of d) is over-expressed.

17. The genetically engineered cell according to claim 14, wherein cell comprises at least two copies, such as at least three copies of the heterologous β-1,3-N-acetyl-glucosaminyl-transferase of i).

18. The genetically engineered cell according to claim 15, wherein the regulatory element for controlling and increasing the expression of i) and ii) is a promoter selected from any one of SEQ ID NO: 12 to 29.

19. (canceled)

20. The genetically engineered cell according to claim 14, wherein the gene product in iii) is the transcriptional repressor GlpR.

21. The genetically engineered cell according to claim 20, wherein the glpR gene of the genetically engineered cell, encoding the DNA-binding transcriptional repressor GlpR, is deleted.

22. A genetically engineered cell according to claim 14, wherein the cell further comprises a recombinant nucleic acid sequence encoding the sugar transport protein YberC or Nec.

23. The genetically engineered cell according to claim 14, wherein the cell is selected from the group consisting of E. coli, C. glutamicum, L. lactis, B. subtilis, S. lividans, P. pastoris, and S. cerevisiae.

24. The genetically engineered cell according to claim 14, which is capable of producing one or more HMOs selected from the group consisting of 2′-FL, LNT-II, LNT, LNFP-I, and DFL.

25. (canceled)


SEQUENCE LISTING (45 sequences)

SEQ ID NO: 1 - protein, 332 aa, Neisseria meningitidis
  MISC_FEATURE (1)..(332): LgtA, -1,3-N-acetyl-glucosaminyltransferase, GeneBank ID WP_033911473.1
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 2 - protein, 439 aa, Artificial Sequence
  galTK, -1,3-galactosyltransferase, homologous to GeneBank ID BD182026.1
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 3 - protein, 292 aa, Sulfuriflexus mobilis
  MISC_FEATURE (1)..(292): Smob, -1,2-fucosyltransferase, GeneBank ID WP_126455392.1
MIISQIIGGLGNQMFQYAAGRALSLVRGQPLLLDVTGFAGYGLHQGFELQRVFDCPIGIATEEDVRGILGWQFSAGIRRIVARPGMAAFRRKGFIVEPHFHYWPEIKNVPRDCYLLGYWQSERYFRAATADIRADFSFKSPLVNRNAETAAQIDQVNAISLHMRRGDYVNNPKTSATHGLCSLDYYQAAIKFVSERVEEPFFFIFSDDIAWVKANLKLDFPCQYVDHNHGAESFNDMHLMSLCQHHIIANSSFSWWGAWLNSDPKKIVLAPKKWFANKNNIKDLFPPGWVSL

SEQ ID NO: 4 - protein, 10 aa, Escherichia coli
  MISC_FEATURE (1)..(10): Lactose permease protein, GeneBank ID
AAAAAAAAAA

SEQ ID NO: 5 - DNA, 999 bp, Artificial Sequence
  lgta coding nucleotide sequence
atgcaaccgctggtctccgtgctgatctgtgcttacaatgtggaaaaatacttcgcccaatcgctggccgcagtcgtgaatcaaacgtggcgcaacctggaaattctgatcgtggatgacggcagtaccgatggtacgctggcgatcgccaaagattttcagaaacgtgactcccgcattaaaatcctggcacaggctcaaaacagtggcctgattccgtccctgaatatcggtctggatgaactggcgaaaagtggcatgggtgaatatatcgcacgcaccgatgctgatgacattgcggccccggactggattgaaaaaatcgtcggcgaaatggaaaaagatcgtagcattatcgcgatgggtgcctggctggaagtgctgtctgaagaaaaagatggcaatcgtctggcacgccatcaccgtcatggtaaaatctggaaaaaaccgacgcgtccggaagatattgccgactttttcccgtttggcaacccgattcacaacaataccatgatcatgcgtcgctcagttattgatggcggtctgcgctataatacggaacgtgattgggcggaagactatcagttctggtacgatgtctcgaaactgggtcgcctggcgtattacccggaagccctggtgaaatatcgtctgcatgccaaccaagttagctctaaatactctatccgccaacacgaaattgcacagggcatccaaaaaaccgctcgtaatgattttctgcagtcaatgggttttaaaacgcgcttcgactcgctggaatatcgtcaaattaaagcggttgcctacgaactgctggaaaaacatctgccggaagaagattttgaacgcgcgcgtcgctttctgtatcagtgcttcaaacgtaccgacacgctgccggcaggtgcttggctggatttcgcagctgacggtcgcatgcgtcgcctgtttaccctgcgtcaatacttcggcattctgcaccgcctgctgaaaaaccgttaa

SEQ ID NO: 6 - DNA, 1320 bp, Artificial Sequence
  GalTK coding nucleotide
atgatctctgtctacatcatcagtctgaaagaatcgcagcgtcgtctggatacggaaaaactggttctggaatcgaacgaaaaatttaaaggccgttgtgtgtttcagattttcgatgcgatctctccgaaacatgaagacttcgaaaaattcgttcaagaactgtacgatagctctagtctgctgaaatcggattggttccatagcgactattgctaccaggaactgctgccgcaagaatttggttgttatctgagccactacctgctgtggaaagaatgcgttaaactgaatcagccggtggttattctggaagatgacgtcgcgctggaatctaactttatgcaggccctggaagattgtctgaaaagtccgtttgacttcgtccgtctgtatggccattactggggcggtcacaaaaccaatctgtgcgcgctgccggtttataccgaaacggaagaagcggaagcctccattgaaaaaaccccgatcgaaaattatgaagtgaccagcccgccgccgccgaacccgacccgcgatacgcagcaagacttcatcaccgaaacgcagcaagatccgaaagaactgtcggaaccgtgcaaaattgccccgcagaaaatcagcttcaaccaagtcgtgttcaagaaaattaaacgtaaactgaaccgcttcatcggtagcatcctggcgcgtaccgaagtctataaaaatatcgtggccaaatacgatgacctgaccacgaaatatgacgatctgaccacgaaatatgatgatctgacgaccaaatatgacgacctgacgacgaaatacgatgacctgaacaaaaacatcgcagaaaaatacgatgaactgatgggcaaatacgaatcgctgctggctaaagaagtgaacatcaaagaaaccttctgggaatcccgtgcggattcagaaaaagaagccctgtttctggaccatttctatctgaccagcgtttacgtcgcaaccacggctggctattacctgaccccgaaaggtgcaaaaaccttcattgaagctacggaacgctttaaaattatcgaaccggttgatatgttcattaacaatccgacctatcatgatattgccaactttacgtacgtgccgtgtccggtttccctgaacaaacacgcattcaactcaaccatccagaacgctaaaaaaccggatattagcctgaaaccgccgaaaaaatcttacttcgataacctgttttatcacaaatttaacgcacgcaaatgcctgaaagcattcaataaatacagtaaacagtacgccccgctgaaaaccccgaaagaagtctaa

SEQ ID NO: 7 - DNA, 879 bp, Artificial Sequence
  smob coding nucleotide sequence
atgatcatcagccagattattggtggtctgggtaatcagatgtttcagtatgcagcaggtcgtgcactgagcctggttcgtggtcagccgctgctgctggatgttaccggttttgcaggttatggtctgcatcagggttttgaactgcagcgtgtttttgattgtccgattggtattgcaaccgaagaagatgttcgcggtattttaggttggcagtttagcgcaggtattcgtcgtattgttgcacgtcctggtatggcagcatttcgtcgtaaaggttttattgtggaaccgcactttcattattggcctgagattaaaaacgttccgcgtgattgttatctgcttggttattggcagagcgaacgttattttcgtgcagcaaccgcagatattcgtgcagatttttcatttaaaagtccgctggttaatcgcaatgccgaaaccgcagcacagattgatcaggttaatgcaattagcctgcatatgcgtcgtggtgattatgtgaataatccgaaaaccagcgcaacccatggtctgtgtagcctggattattatcaggcagcaatcaaatttgttagcgaacgtgttgaagaaccgtttttctttatcttctccgatgatattgcatgggtgaaagcaaatctgaaactggattttccgtgccagtatgtggatcataatcatggtgcagaaagcttcaatgatatgcatctgatgagcctgtgtcagcatcatattattgcaaacagcagctttagttggtggggtgcatggctgaatagcgatccgaaaaaaatcgttctggcaccgaaaaaatggttcgccaacaaaaacaacatcaaagacctgtttccgcctggttgggttagcctgtaa

SEQ ID NO: 8 - protein, 302 aa, Artificial Sequence
  FutC, -1,2-fucosyltransferase, homolouge to GeneBank ID WP_080473865.1
MAFKVVQICGGLGNQMFQYAFAKSLQKHSNTPVLLDITSFDWSDRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYQCKLSLILAAKNSVFVHIRRGDYVGIGCQLGIDYQKKALEYMAKRVPNMELFVFCEDLEFTQNLDLGYPFMDMTTRDKEEEAYWDMLLMQSCQHGIIANSTYSWWAAYLIENPEKIIIGPKHWLFGHENILCKEWVKIESHFEVKSQKYNALG

SEQ ID NO: 9 - DNA, 909 bp, Artificial Sequence
  futC coding nuelcotide sequence
atggcgttcaaagtggtccaaatctgcggtggtctgggtaatcaaatgttccaatatgccttcgctaaatcgctgcaaaaacacagtaataccccggtcctgctggatattacgagttttgattggtccgaccgtaaaatgcagctggaactgttcccgattgatctgccgtatgcgagcgccaaagaaatcgcaattgctaaaatgcagcatctgccgaaactggttcgtgatgcgctgaaatgcatgggctttgaccgcgtcagtcaagaaatcgtgttcgaatatgaaccgaaactgctgaaaccgtcccgtctgacctatttctttggttactttcaggacccgcgttacttcgacgccatctctccgctgattaaacaaacctttacgctgccgccgccgccggaaaacaacaaaaacaacaacaaaaaagaagaagaatatcagtgcaaactgagcctgatcctggcggccaaaaactctgtgtttgttcacattcgtcgcggcgattacgtgggcatcggttgtcagctgggtattgactatcagaaaaaagcgctggaatacatggccaaacgtgttccgaatatggaactgtttgtcttctgcgaagatctggaatttacccaaaacctggacctgggctatccgttcatggatatgaccacgcgcgacaaagaagaagaagcgtattgggatatgctgctgatgcagagctgtcaacatggtattatcgctaatagcacgtattcttggtgggcagcttacctgattgaaaacccggaaaaaattatcattggcccgaaacattggctgtttggtcacgaaaatatcctgtgtaaagaatgggtgaaaatcgaatcacacttcgaagttaaatcgcagaaatataacgcgctgggctaa

SEQ ID NO: 10 - DNA, 1254 bp, Escherichia coli
  misc_feature (1)..(1254): LacY encoding nucleotide sequence
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

SEQ ID NO: 11 - DNA, 152 bp, Klebsiella pneumoniae
  misc_feature (1)..(152): Promoter for scrYA sucrose genes
ggttaacggcccactttgctggcgacatcacaattcttaaaccggtttagcaatttttattttcaccgcgttaccgacatgtttaccatatcaactaaaccggtttagcaaacattagcacactcactgatttacctttggatgtcaccaac

SEQ ID NO: 12 - DNA, 291 bp, Artificial Sequence
  PgatY_70UTR, variant of E. coli promoter for gatYZABCD; tagatose-1,6-bisP aldolase
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgtgcctacaagcatcgtggaggtccgtgactttcacgcatacaacaaacattaaccaaggaggaaacagct

SEQ ID NO: 13 - DNA, 300 bp, Escherichia coli
  misc_feature (1)..(300): PglpF, E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 14 - DNA, 300 bp, Artificial Sequence
  PglpF_SD1, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 15 - DNA, 300 bp, Artificial Sequence
  PglpF_SD10, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 16 - DNA, 300 bp, Artificial Sequence
  PglpF_SD2, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagcgcaaaacagct

SEQ ID NO: 17 - DNA, 300 bp, Artificial Sequence
  PglpF_SD3 variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 18 - DNA, 300 bp, Artificial Sequence
  PglpF_SD4, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 19 - DNA, 300 bp, Artificial Sequence
  PglpF_SD5, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 20 - DNA, 300 bp, Artificial Sequence
  PglpF_SD6, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 21 - DNA, 300 bp, Artificial Sequence
  PglpF_SD7, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 22 - DNA, 300 bp, Artificial Sequence
  PglpF_SD8, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 23 - DNA, 300 bp, Artificial Sequence
  PglpF_SD9, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 24 - DNA, 300 bp, Artificial Sequence
  PglpF_B29, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgatttaattacagcatgcctacaagcatcgtggaggtccgtgactttcacgcatacaacaaacattaaccaaggaggaaacagct

SEQ ID NO: 25 - DNA, 300 bp, Artificial Sequence
  PglpF_B29, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgatcagaatacagcatgcctacaagcatcgtggaggtccgtgactttcacgcatacaacaaacattaaccaaggaggaaacagct

SEQ ID NO: 26 - DNA, 107 bp, Artificial Sequence
  Plac_16UTR, variant of E. coli lac operon promoter
tgtgagttagctcactcattaggcaccccaggctttacactttatgcttccggctcgtatgttgtgtggaattgtgagcggataacaatttcaaggaggaaacagct

SEQ ID NO: 27 - DNA, 107 bp, Escherichia coli
  misc_feature (1)..(107): Plac, lac operon promoter
tgtgagttagctcactcattaggcaccccaggctttacactttatgcttccggctcgtatgttgtgtggaattgtgagcggataacaatttcacacaggaaacagct

SEQ ID NO: 28 - DNA, 203 bp, Artificial Sequence
  PmglB_70UTR, variant of E. coli promoter for mglBAC galactose/methyl-galactosidade transporter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 29 - DNA, 203 bp, Artificial Sequence
  PmglB_70UTR_SD4, variant of E. coli promoter for mglBAC; galactose/methyl-galactosidade transporter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 30 - protein, 505 aa, Klebsiella pneumoniae
  MISC_FEATURE (1)..(505): ScrY, sucrose porin, GeneBank ID CAA40657.1
MYKKRKLAILIALLTGTAAAHGQTDLNSIEARLAALEKRLQDAETRASTAESRAASAEQKVQQLTQQQQQTQATTQQVARRTTQLEEKAERPGGFEFHGYARSGVIMNDSAASTKSGAYMTPAGETGGAIGRLGNQADTYVEMNLEHKQTLDNGATTRFKVMVADGQTTYNDWTASSSDLNVRQAFVELGNLPTFEGPFKGSTLWAGKRFDRDNFDIHWIDSDVVFLAGTGGGIYDVKWNDSLRSNFSLYGRNFGDIADSSNSVQNYIVSMNNFAGPVQMMVSGMRAKDNDDRQDANGNLVKGDAANTGVHALLGLHNESFYGLRDGTSKTALLYGHGLGAEVKGIGSDGALRPGANTWRFASYGTTPLSDRWFIAPAVLAQSSKDRYVDGDSYQWATLNLRLIQEVTQNFALAWEGSYQYMDLQPEGYNDRHAVNGSFYKLTFAPTFKVGSIGDFFSRPEIRFYTSWMDWSKKLDNYANDDALGSNGFKSGGEWSFGMQMETWF

SEQ ID NO: 31 - protein, 456 aa, Klebsiella pneumoniae
  MISC_FEATURE (1)..(456): ScrA, sucrose-specific enzyme II, GeneBank ID CAA40658.1
MDFEQISRSLLPLLGGKENIASAAHCATRLRLVLVDDALADQQAIGKIDGVKGCFRNAGQMQIIFGTGVVNKVYAAFIQAAGISESSKSEAADLAAKKLNPFQRIARLLSNIFVPIIPAIVASGLLMGLLGMVKTYGWVDPSNALYIMLDMCSSAAFIILPILIGFTAAREFGGNPYLGATLGGILTHPALTNAWGVAAGFHTMNFFGIEVAMIGYQGTVFPVLLAVWFMSMVEKRLRRVIPDALDLILTPFLTVIISGFIALLLIGPAGRALGDGISFILSTLISHAGWLAGLLFGGLYSVIVITGIHHSFHAIEAGLLGNPSIGVNFLLPIWAMANVAQGGACFAVWFKTKDAKIKAITLPSAFSAMLGITEAAIFGINLRFVKPFIAALVGGAAGGAWVVSMHVYMTAVGLTAIPGMAIVQASSLLNYIIGMAIAFAVAFALSLTLKYKTDAE

SEQ ID NO: 32 - protein, 466 aa, Salmonella enterica subsp. enterica serovar Typhimurium
  MISC_FEATURE (1)..(466): ScrB, beta-fructofuranosidase, GeneBank ID CAA47974.1
MSLPSRLPAILQAVMQGQPRALADSHYPRWHHAPVTGLMNDPNGFIEFAGRYHLFYQWNPLACDHTFKCWAHWSSIDLLHWQHEPIALMPDEEYDRNGCYSGSAVDNNGTLTLCYTGNVKFAEGGRTAWQCLATENADGTFRKIGPVLPLPEGYTGHVRDPKVWRHEDLWYMVLGAQDRQKRGKVLLFSSADLHQWTSMGEIAGHGINGLDDVGYMWECPDLFPLGDQHILICCPQGIAREEECYLNTYPAVWMAGEFDYAAGAFRHGELHELDAGFEFYAPQTMLTSDGRRLLVGWMGVPEGEEMLQPTLNNGWIHQMTCLRELEFINGQLYQRPLRELSALRGEANGWSGNALPLAPMEIDLQTRGGDMLSLDFGGVLTLECDASGLRLARRSLASDEMHYRYWRGNVRSLRVFIDQSSVEIFINGGEGVMSSRYFPACSGQLTFSGITPDAFCYWPLRTCMVE

SEQ ID NO: 33 - protein, 334 aa, Salmonella enterica subsp. enterica serovar Typhimurium
  MISC_FEATURE (1)..(334): ScrR, sucrose repressor, GeneBank ID CAA47975.1
MKTKRVTIKDIAEQAGVSKATASLVLNGRGKELRVAQETRERVLSIARKHHYQPSIHARSLRNNRSHTIGLVVPEITNHGFAVFAHELEMLCREAGVQLLISCTDENPGQESVVVNNMIARQVDGMIVASCMHNDADYLKLSQQLPVVLFDRCPNESALPLVMTDSITPTAELISRIAPQHSDEFWFLGGQARLSPSRDRLTGFTQGLAQAGIALRPEWVINGNYHPSSGYEMFAALCARLGRPPKALFTAACGLLEGVLRYMSQHHLLDSDIHLTSFDDHYLYDSLSLRIDTVQQDNRQLAWHCYDLISQLIEGDTPETLQRYLPATLQFRHQ

SEQ ID NO: 34 - protein, 483 aa, Avibacterium gallinarum
  MISC_FEATURE (1)..(483): SacC_AgaI, glycoside hydrolase family 32 protein, GeneBank ID WP_103853210.1
MIIFNEGKYKSLYAAEQGELEKIAQTVAQDQDFRPVYHLAPPTGLLNDPNGLIFDGEKYHLFYQWYPFDALHGMKHWQHFITQDFKQFSQADLLVPCELYESHGCYSGGAVKIGDQIAVFYTGNTRRPSDNQRVPYQNLAIFSKDGKLLSKRPLIEQAPQGYTEHVRDPKPFLTKDGKIRFICGAQRENLTGTALVFEMDNLADTPRLLGELALPAFDNQGVFMWECPDLSQMGDKSLFIWSPQGKARELEQYQNNYHAVYALGELADRQFHAEQIAELDQGFDFYAPQTFSGTQTMLLGWVGLPDLSYPTDLYKWHSMLSMPRQLRLQDGKIYQQPIENIYKNLTALQSITVEKEAEIADLDRAYLKFDANAQPFSLKFFNNAQNQRLILSYDGEMLCLDRSQTEQTDSMKSFGDKRYCRIEDLRQVEIFFDRSVAEIFLNQGEKAMTSRFFICARENQLCTDKPLTLQVGYPKKIEVDYTK

SEQ ID NO: 35 - protein, 548 aa, Arthrobacter globiformis
  MISC_FEATURE (1)..(548): Bff, beta-fructofuranosidase protein, GeneBank ID BAD18121.1
MERTCITVRAIVRFHIEQRQTIVNKQRTKRGILTAALSIGALGATLISGPAVAATDAAPGFPQPTEHTQKAYSPTDNFTSRWTRADAKQLKAMSDPDAGSRENSMPTEYTMPTVSQDFPDMSNEKVWVWDTWPLIDENANQYSVNGQEIIFSLVADRKLGFDERHQYARIGYFYRPAGIPADERPEDGGWTYGGQVFDEGVTGKIFEDQSFTHQTQWSGSARVSKNGEIKLFFTDVAFYRDKDGQDVKPYDSRIALSVGHVHSNKKGVKLTGFNKVKELLQADGKNYQNAAQNSYYNFRDPFTFVDPAHPGETYMVFEGNSAMDRDEAKCTAEDLGYREGETNGETVEQVNNSGATYQIGNVGLARAKNKALTEWEFLPPILSANCVTDQTERPQIYMQDGKYYLFTISHRSTFATGIDGPEGVYGFVGNGIRSDYQPLNRGSGLALGSPTNLNFAAGTPFAPDYNQHPGQFQAYSHYVMPGGLVQSFIDTIGTKDNFVRGGTLGPTVKLNIKGDSATVDYNYGDNGLGGWADIPANRELKNSKAVAK

SEQ ID NO: 36 - DNA, 152 bp, Artificial Sequence
  Pscr_SD1 promoter sequence, variant of Klebsiella pneumoniae Pscr Promoter
ggttaacggcccactttgctggcgacatcacaattcttaaaccggtttagcaatttttattttcaccgcgttaccgacatgtttaccatatcaactaaaccggtttagcaaacattagcacactcactgatttacccaaattcgaaacagct

SEQ ID NO: 37 - DNA, 152 bp, Artificial Sequence
  Pscr_SD7 promoter sequence, variant of Klebsiella pneumoniae Pscr Promoter
ggttaacggcccactttgctggcgacatcacaattcttaaaccggtttagcaatttttattttcaccgcgttaccgacatgtttaccatatcaactaaaccggtttagcaaacattagcacactcactgatttacccaagagcaaaacagct

SEQ ID NO: 38 - protein, 387 aa, Rouxiella badensis
  MISC_FEATURE (1)..(387): Bad MFS transporter
MSSRRLSIIFATFLLVSFLTGIAGALQAPTLSLFLTNEVKVRPLWVGLFYTVNALGGIVISFLLANYSDKKGDRRKLLFFCTLMAIGNSLIFAYSRDYLVLISVGVLLAAIGNASMPQLFALAREYADRSAHEVVMFSSMMRATLSLAWVLGPPISFTLALNYGFTLMYLCAAGVFIFSALMVWFFLPSVGRIEQPVDKVVVHVSAWKNRDVRLLFFASLLMWTCNIMYIIDMPLYITSDLGLPEGLAGLLMGAAAGLEIPVMLIAGYLVKRTGKRRLMLCAAVFGILFYLGLVLFQFKAALMILQLFNAIFIGIIAGIGMLYFQDLMPGRAGSATTLFTNSISTGAILAGVIQGTIVQNFGHYQVYWMALALAVGALVLMTRVKNV

SEQ ID NO: 39 - protein, 394 aa, Rosenbergiella nectarea
  MISC_FEATURE (1)..(394): Nec MFS transporter
MQSFTPPAPKGGNPVFMMFMLVTFFVSIAGALQAPTLSLYLSQELAAKPFMVGLFFTINAVTGIIISFILAKRSDRKGDRRRLLMFCCAMAIANALMFAFVRQYVVLITLGLILSALTSVVMPQLFALAREYADRTGREVVMFSSVMRTQMSLAWVIGPPISFALALNYGFITLYLVAAALFLLSLILIKTTLPSVPRLYPAEDLAKSAASGWKRTDVRFLFAASVLMWVCNLMYIIDMPLYISKSLGMPESFAGVLMGTAAGLEIPVMLLAGYLAKRVGKRPLVIVAAVCGLAFYPAMLVFHQQTGLLIIQLLNAVFIGIVAGLVMLWFQDLMPGKAGAATTLFTNSVSTGMIFAGLCQGLLSDLLGHQAIYVLATVLMVIALLLLLRVKEQA

SEQ ID NO: 40 - protein, 393 aa, Yersinia bercovieri
  MISC_FEATURE (1)..(393): YberC MFS transporter
MKSALTFSRRINPVFLAFFVVAFLSGIAGALQAPTLSLFLSTEVKVRPLWVGLFYTVNAIAGITVSFILAKRSDSRGDRRKLIMVCYLMAVGNCLLFAFNRDYLTLITAGVLLASVANTAMPQIFALAREYADSSAREVVMFSSIMRAQLSLAWVIGPPLSFMLALNYGFTLMFSIAAGIFVLSALVVWFILPSVPRAEPVVDAPVVVQGSLFADKNVLLLFIASMLMWTCNTMYIIDMPLYITASLGLPERLAGLLMGTAAGLEIPIMLLAGYSVRYFGKRKIMLFAVLAGVLFYTGLVLFKFKTALMLLQIFNAIFIGIVAGIGMLYFQDLMPGRAGAATTLFTNSISTGVILAGVLQGGLTETWGHDSVYVMAMVLSILALIICARVREA

SEQ ID NO: 41 - protein, 393 aa, Yersinia frederiksenii
  MISC_FEATURE (1)..(393): Fred MFS transporter
MKSALTFSRRINPVFLAFFVVAFLSGIAGALQAPTLSLFLSTEVKVRPLWVGLFYTVNAIAGITVSFVLAKRSDLRGDRRKLILVCYLMAVGNCLLFAFNRDYLTLITAGVLLAAVANTAMPQIFALAREYADNSAREVVMFSSIMRAQLSLAWVIGPPLSFMLALNYGFTLMFCIAAGIFVLSALVVWFILPSVQRAEPVMDAPTVAQGSLFADKDVLLLFIASMLMWTCNTMYIIDMPLYITASLGLPERLAGLLMGTAAGLEIPIMLLAGYSVRRFGKRKIMLFAVLAGVLFYTGLVLFKFKSALMLLQIFNAIFIGIVAGIGMLYFQDLMPGRAGAATTLFTNSISTGVILAGVLQGVLTETWGHNSVYVMAMILAILSLIICARVREA

SEQ ID NO: 42 - protein, 392 aa, Pantoea vagans
  MISC_FEATURE (1)..(392): Vag MFS transporter
MKSLLTRKRRINPVFLAFMAASFMIGVAGALQAPTLSLFLTREVQARPLWVGLFFTVNAIAGIVVSMLVAKRSDSRGDRRTLILFCCAMAFCNALLFAFTRHYLTLITLGVLLSALASVSMPQIFALAREYADQSAREAVMFSSVMRAQLSLAWVIGPPLSFALALNFGFVTLFLVAAALFLVCILLIKFTLPSVPRAEPLMRSGGMPLSGWRDRDVRLLFIASVTMWTCNTMYIIDMPLYISVTLGLPEKLAGLLMGTAAGLEIPVMLLAGHYAKRVGKRNLMLIAVAAGVLFYAGLAMFASQTALMALQLFNAVFIGIIAGIGMLWFQDLMPGRPGAATTMFTNSISTGMILAGVIQGTLSERFGHIAVYWLALGLAVAAFAMSARVKNV

SEQ ID NO: 43 - protein, 398 aa, Serratia marcescens
  MISC_FEATURE (1)..(398): Marc MFS transporter
MQRLSRLSLRINPIFAAFLLIAFLSGIAGALLTPTLSLFLTTEVKVRPLWVGLFYTANAVAGIVVSFLLAKRSDTRGDRRRLILLCCLMAVGNCLLFAFNRDYLTLITAGVLMSAVANTAMPQIFALAREYADSEAREVVMFSSVMRAQLSLAWVIGPPLSFALALNYGFTVMFLIAAVTFAVCVLLVGFMLPSVPRAAENEGLQGGVSAPIAPASAWRNRDVRLLFIASMLMWTCNTLYIIDMPLYITADLGLPEGLAGVLMGTAAGLEIPAMLLAGYYVKRFGKRNMMLLAVVAGVLFYLGLTVLESKPALIALQLLNAVFIGIVAGIGMLYFQDLMPGRPGAATTLFTNSISTGVILAGVLQGALVENLGHGSVYWMAALLALAALGMSAKVREV

SEQ ID NO: 44 - DNA, 759 bp, Escherichia coli
  misc_feature (1)..(759): glpR gene
atgaaacaaacacaacgtcacaacggtattatcgaactggttaaacagcagggttatgtcagtaccgaagagctggtagagcatttctccgtcagcccgcagactattcgccgcgacctcaatgagctggcggagcaaaacctgatcctgcgccatcatggcggtgcggcgctgccttccagttcggttaacacgccgtggcacgatcgcaaggccacccagaccgaagaaaaagagcgcatcgcccgcaaagtggcggagcaaatccccaatggctcgacgctgtttatcgatatcggcaccacgccggaagcggtagcgcacgcactgctcaatcacagcaatttgcgcattgtcaccaacaatctcaacgttgctaacacgttgatggtaaaagaagattttcgcatcattctcgccggtggcgaattacgcagccgcgatggcgggatcattggcgaagcgacgctcgattttatctcccagtcccgccttgatttcggcattctggggataagcggcatcgatagcgacggctcgctgctggagttcgattaccacgaagttcgcaccaaacgcgccattattgagaactcgcgccacgttatgctggttgtcgatcactcgaaatttggccgtaacgcgatggtcaatatgggcagcatcagcatggtagatgccgtctacaccgacgccccgccgccagtaagcgtgatgcaggtgctgacggaccaccatattcaactggagctgtgctga

SEQ ID NO: 45 - DNA, 6706 bp, Escherichia coli
  misc_feature (1)..(6706): CA gene cluster
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa